(19) United States
(12) Patent Application Publication
(10) Pub. No.: US 2023/0017597 A1
(43) Pub. Date: Jan. 19, 2023

(54) 8-SUBSTITUTED STYRYL XANTHINE DERIVATIVES AND USES THEREOF

(71) Applicant: SUNSHINE LAKE PHARMA CO., LTD., Dongguan, Guangdong (CN)

(72) Inventors: Chuanfei JIN, Dongguan (CN); Wenhe ZHONG, Dongguan (CN); Tengfei XU, Dongguan (CN)

(73) Assignee: SUNSHINE LAKE PHARMA CO., LTD., Dongguan, Guangdong (CN)

(21) Appl. No.: 17/773,374

(22) PCT Filed: Nov. 9, 2020
(86) PCT No.: PCT/CN2020/127456

§ 371 Date: Apr. 29, 2022

(30) Foreign Application Priority Data
Nov. 11, 2019 (CN) 201911094929.4

(51) Int. Cl. A61K 31/522; A61K 31/198; C07D 473/06

(52) U.S. Cl. CPC A61K 31/522; A61K 31/198; C07D 473/06

(57) ABSTRACT

An 8-substituted styryl xanthine derivatives and uses thereof, specifically, relates to a novel 8-substituted styryl xanthine derivative and a pharmaceutical composition containing this compound, which can be selective adenosine A_{2A} receptor antagonist, and also relates to methods of preparing this compound and pharmaceutical composition, and uses thereof in the manufacture of a medicament for treating an adenosine A_{2A} receptor-related disease, especially Parkinson's Disease.

### CROSS-REFERENCE TO RELATED APPLICATION

This application claims priority and benefits of Chinese Patent Application No. 201911094929.4, filed with State Intellectual Property Office on 11 Nov. 2019, the entire content of which is incorporated herein by reference.

### FIELD OF THE INVENTION

The invention pertains to the pharmaceutical field, specifically relates to novel 8-substituted styryl xanthine derivatives and pharmaceutical compositions containing these compounds, and usage methods and uses thereof. Specially, the novel 8-substituted styryl xanthine derivative of the present invention can be used as a selective adenosine A_{2A} receptor antagonist for preventing, treating or lessening an adenosine A_{2A} receptor-related disease, especially Parkinson's disease.

### BACKGROUND OF THE INVENTION

Parkinson's disease (PD) is a common chronic degenerative disease of the nervous system, also known as tremor paralysis. The elderly are more common, and the average age of onset is about 60 years old. Parkinson's disease is less common in young people under the age of 40. The prevalence of PD in China over 65 years old is about 1.7%. Most patients with Parkinson's disease are sporadic cases, and less than 10% have a family history. Parkinson's disease is insidious, and progresses slowly. The first symptom is usually the tremor or awkwardness of one limb, which in turn affects the contralateral limb. The main clinical manifestations are resting tremor, bradykinesia, myotonia and posture gait disorder. In recent years, more and more people have noticed that non-motor symptoms such as depression, constipation and sleep disorders are also common complaints of patients with Parkinson's disease. Their influence on patients' quality of life even exceeds the symptoms of exercise. PD seriously affects patients' daily life and social activities, and has become a major disease that affects people, affecting the quality of life of millions of people around the world.

The most important pathological change of Parkinson's disease is the degenerative death of dopamine (DA) neurons in the midbrain, which causes a significant decrease in the striatum DA content and causes disease. The exact cause of this pathological change is still unclear. Genetic factors, environmental factors, aging, and oxidative stress may be involved in the degenerative death of PD dopaminergic neurons.

The current treatment of PD mainly includes surgical treatment and drug treatment. Surgical treatment may have serious adverse reactions and high postoperative recurrence rate, which limits the widespread application of surgical treatment (Kelly P J, Gillingham F J. The long-term results of stereotaxic surgery and L-dopa therapy in patients with Parkinson's disease. A 10-year follow-up study [J]. J Neurosurg, 1980, 53(3): 332-7). The drug treatment of PD is mainly divided into two categories: anticholinergic drugs and drugs that affect dopaminergic. Anticholinergic drugs can only improve symptoms as an adjunct. Dopaminergic treatment for PD is mainly to reverse the lack of striatal dopamine caused by nigrostriatal damage (Obeso J A, Olanow C W, Nutt J G Levodopa motor complications in Parkinson's disease [J]. Trends Neurosci, 2000, 23(10):2-7). Levodopa and other dopamine agonists are effective in controlling the symptoms of PD, especially in the early stages of the disease. However, dopamine agonists have acute side effects such as hypotension, nausea, vomiting, and other symptoms that occur with the severity of the disease, including loss of drug effectiveness, psychosis, and dyskinesia. It can be seen that the current treatment is symptomatic and does not significantly improve the progress of the disease, so there is an urgent need to develop other methods for treating PD. New PD treatments should be effective throughout the disease, not only reducing the side effects of existing treatments, but also having neuroprotective effects that slow or prevent the progression of the disease.

Many of the newly studied drugs for the treatment of PD are mainly non-dopaminergic systems targeting the basal ganglia, which have strong anti-PD activity and no side effects.

The basal ganglia is an important subcortical center that regulates movement, and mainly includes two pathways: direct pathway (striatal-substantia nigra reticulate/globus pallidus internus-thalamus-cortical loop), indirect pathway (striatum-lateral globus pallidus-subthalamic nucleus-substantia nigra reticulum/medial paleosphere-thalamus-cortex circuit). The striatum acts on an efferent neuron in the direct pathway that contains dopamine D₁ receptors, and the striatum acts on efferent neurons of the indirect pathway, which contains dopamine D₂ receptors. Activation of the direct pathway facilitates thalamic cortical neuron activity, while activation of the indirect pathway inhibits thalamic cortical neuron activity. Dopamine has an excitatory effect on the D₁ receptor to activate the direct pathway, and an inhibitory effect on the D₂ receptor to inhibit the indirect pathway, keeping the direct and indirect pathways in balance. After DA neurons in the substantia nigra compaction of Parkinson's disease are injured, the activation of direct pathways and the inhibition of indirect pathways weaken, leading to imbalance between direct and indirect pathways, and the inhibition of thalamic cortical neurons is strengthened. Stiffness, tremor, bradykinesia, reduced movement and the like appear (Lang A E. Lozano A M. Parkinson's disease. Second of two parts. New England Journal of Medicine, 1998, 339(16):1130-1143).

Adenosine A_{2A} receptor is selectively expressed in basal ganglia and is related to motor behavior, mainly acts through the regulation of indirect pathways: (1) activating adenosine A_{2A} receptors of GABAergic neurons in the striatum can increase the excitability of GABAergic neurons in the striatum, thereby inhibiting the excitability of GABAergic neurons in external globus pallidus; (2) activating adenosine A_{2A} receptors at the axon end of the GABAergic neurons in the striatum can promote GABA release, inhibit the excitability of GABAergic neurons in external globus pallidus (Shindou T, Richardson P J, Mori A et, al. Adenosine modulates the striatal GABAergic inputs to the globus pallidus via adenosine A_{2A} receptors in rats. Neuroscience Letters, 2003, 352(3):167-170.)

Epidemiological and laboratory studies have shown that blocking adenosine A_{2A} receptors can alleviate degenerative changes in dopaminergic neurons. Adenosine A_{2A} receptor antagonists improve PD symptoms while slowing disease progression. Therefore, adenosine A_{2A} receptor antagonist as non-dopamine target of basal ganglia may develop into new strategy for treating PD (Pinna A, Wardas J, Simola N, et, al. New therapies for the treatment of Parkinson's disease: Adenosine A_{2A} receptor antagonists [J]. LifeSci, 2005, 77(26):3259-67). Numerous basic and clinical studies suggest that adenosine A_{2A} receptor antagonists may become a new class of drugs for treating Parkinson's disease. How to find some drugs that have high affinity for adenosine A_{2A} receptors, can also play a good role in the body, and have few adverse reactions has become an important topic in the research of adenosine A_{2A} receptor antagonists.

Adenosine receptors represent a subclass of receptors of the coupling of purine nucleotides and nucleoside G protein receptors (called purine receptors). There are four major adenosine receptor subtypes that differ in pharmacology, i.e. A₁, A_{2A}, A_{2B}, and A₃, respectively. The major adenosine receptor subtypes in the brain are A₁ and A_{2A}. However, it was found that adenosine A₁ receptors are distributed throughout the brain at high density, and the distribution of adenosine A_{2A} receptors is more restricted. Adenosine A_{2A} receptors are present in the striatum (olfactory nodules, nucleus accumbens, lateral caudate nucleus) at high density, and are localized together with dopamine D₂ receptors on striatum efferent neurons. Adenosine A_{2A} receptors are mainly co-expressed with dopamine D₂ receptors in the medium spiny neurons (MSNs) of striatum-globus pallidus, but not co-expressed with dopamine D₁ receptors (Fink J S, Weaver D R, Rivkees S A et, al. Molecular Cloning of the rat A₂ adenosine receptor: Selective co-expression with D₂ dopamine receptors in rat striatum. Brain Res Mol BRAIN rES, 1992, 14:186-195). Later, it was found that adenosine A_{2A} receptor and dopamine D₂ receptor were also co-expressed in the core and shell regions of olfactory nodules and the nucleus accumbens (Svenningsson P, Le Moine C, Kull B et, al. Cellular expression of adenosine A_{2A} receptor messenger RNA in the rat central nervous system with special reference to dopamine inervetd areas. Neuroscience, 1997, 80:1171-1185), and also distributed in peripheral parts such as immune cells (Sitkovsky M V, Lukashev D, Apasov S et, al. Physiological control of immune response and inflammatory tissue damage by hypoxia-inducible factors and adenosine A_{2A} receptors. Annual Review of Immunology, 2004, 22:657-682). The discrete localization of adenosine A_{2A} receptors in the striatum and their ability to functionally antagonize D₂ receptors have made adenosine A_{2A} receptor antagonists a potential therapy for Parkinson's symptoms.

Studies (Fuxe K, Ferre S, Canals M et, al. Adenosine A_{2A} and dopamine D₂ heteromeric receptor complexes and their function. Journal of Molecular Neuroscienci, 2005, 26(2-3):209-220) have shown that adenosine A_{2A} receptors and dopamine D₂ receptors can form heterodimer and/or hetero-oligomer, wherein heterodimer can reduce the activity of dopamine D₂ receptor: through the interaction between the carboxyl terminus of the adenosine A_{2A} receptor and the 5,6 transmembrane region of the dopamine D₂ receptor, the epitope of the dopamine D₂ receptor is changed, and the affinity of dopamine D₂ receptor and its ligand is reduced; through the interaction between the carboxyl terminus of the adenosine A_{2A} receptor and N-terminal Part of I3 of the D₂ receptor (arginine-rich epitope), the coupling of dopamine D₂ receptor and G protein is reduced, the effect of dopamine D₂ receptor activation to promote K⁺ outflow and inhibit calcium inflow is reduced, thereby the activity of dopamine D₂ receptor is reduced. Adenosine A_{2A} receptor agonists can promote the formation of heterodimer, while adenosine A_{2A} receptor antagonists can inhibit the formation of heterodimer.

Naturally occurring xanthine is the first generation of adenosine receptor antagonists, such as caffeine (1,3,7-caffeine) and theophylline (1,3-dimethylxanthine, Daly et, al., Cell. Mol. Neurobiol., 1983, 3, 67). These xanthines have long been known to reverse motor deficits in various PD models. Moreover, epidemiological investigations suggest that caffeine and theophylline can reduce the incidence of Parkinson's disease. However, research (Fredholm B B. Connection between caffeine, adenosine receptors and dopamine. Coffee reduces the risk of Parkinson's disease. Lakartidningen, 2004, 101(34):2552-2555) has found that coffee is a non-selective adenosine A_{2A} receptor antagonist that works by blocking the adenosine A_{2A} receptor. Because they are non-selective and moderately effective, they have led to the development of selective adenosine A_{2A} receptor antagonists.

It was further found through various synthetic substitutions on the xanthine moiety that the introduction of styryl at position 8 of xanthine is essential in obtaining compounds with antagonistic effect on selective adenosine A_{2A} receptor (Ongini et, al., Trends Pharmacol. Sci., 1996, 17, 364; Shimada et, al., J. Med. Chem., 1992, 36, 2343; Muller et, al., Curr. Pharm. Des., 1996, 2, 501; Baraldi et, al., Curr. Med. Chem., 1995, 2, 707). This work found structurally related compound KF17837 ((E)-1,3-dipropyl-8-(3,4-dimethoxystyryl)-7-methylxanthine) and its analogue KW6002 (istradefylline, (E)-1,3-diethyl-8-(3,4-dimethoxystyryl)-7-methylxanthine), whose pharmacological properties have been extensively studied. Despite similarity in vitro potency, these two structurally similar xanthines appear to have significant differences in potency in vivo, such as measuring systemic stiffness in mice by attenuation, of which KW6002 is significantly more effective. This difference in activity in vivo may be due to differences in pharmacokinetics, pharmacodynamics, metabolism and/or bioavailability (Kiec-Kononowicz et, al., Pure and Appl. Chem., 2001, 73, 1411).

Adenosine A_{2A} receptor antagonist, as a new drug for the treatment of PD, has accurate effects, good safety and good tolerance, and has broad application prospect.

### SUMMARY OF THE INVENTION

Specially, the present invention provides a novel 8-substituted styryl xanthine derivative as a selective adenosine A_{2A} receptor antagonist for treating an adenosine A_{2A} receptor-related disease, especially Parkinson's disease. Through experiments, it was found that the 8-substituted styryl xanthine derivative has stable properties, good safety, favorable pharmacodynamics and good pharmacokinetic properties, such as expected brain/plasma ratio, good bioavailability or good metabolic stability, and so on. Therefore, it has a good clinical application prospect.

The invention also provides a method for preparing the compound and a pharmaceutical composition containing the compound, and uses of the compound and the pharmaceutical composition containing the compound in the manufacture of a medicament.

In one aspect, provided herein is a compound having formula (I) or a stereoisomer, a tautomer, an N-oxide, a hydrate, a solvate, a metabolite, a pharmaceutically acceptable salt or a prodrug thereof,

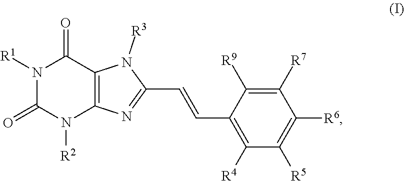

wherein

each of R¹, R², and R³ is independently H, D, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, hydroxyl substituted C₁-C₆ alkyl, C₃-C₈ cycloalkyl, 3-8 membered heterocyclyl, C₆-C₁₀ aryl or 5-10 membered heteroaryl;

each of R⁴, R⁵ and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxyl substituted C₁-C₆ alkyl, C₃-C₈ cycloalkyl, 3-8 membered heterocyclyl, C₆-C₁₀ aryl or 5-10 membered heteroaryl;

R⁶ is —O—R⁰, R⁷ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxyl substituted C₁-C₆ alkyl, C₃-C₈ cycloalkyl, 3-8 membered heterocyclyl, C₆-C₁₀ aryl, 5-10 membered heteroaryl or —O—R⁰; or

R⁶ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxy substituted C₁-C₆ alkyl, C₃-C₈ cycloalkyl, 3-8 membered heterocyclyl, C₆-C₁₀ aryl or 5-10 membered heteroaryl, R⁷ is —O—R⁰;

R⁰ is

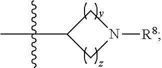

y is 1, 2, or 3;

z is 1, 2, or 3; and

R⁸ is H, D, —COOH, —C(═O)NH₂, —C(═O)NHCH₃, —C(═O)N(CH₃)₂, —C(═O)—(C₁-C₆ alkyl), —C(═O)—(C₃-C₈ cycloalkyl), —C(═O)-(3-8 membered heterocyclyl), —C(═O)—(C₆-C₁₀ aryl), —C(═O)-(5-10 membered heteroaryl), —C(═O)—(C₁-C₆ alkoxy), —S(═O)₂—(C₁-C₆ alkyl), —S(═O)₂—(C₃-C₈ cycloalkyl), —S(═O)₂-(3-8 membered heterocyclyl), —S(═O)₂—(C₆-C₁₀ aryl), —S(═O)₂-(5-10 membered heteroaryl), C₁-C₆ alkyl, C₂-C₆ alkenyl, C₂-C₆ alkynyl, C₁-C₆ haloalkyl, hydroxy substituted C₁-C₆ alkyl, C₃-C₈ cycloalkyl, 3-8 membered heterocyclyl, C₆-C₁₀ aryl or 5-10 membered heteroaryl.

In some embodiments, R⁰ is

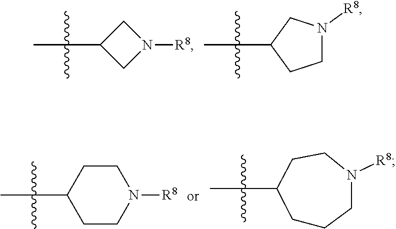

wherein R⁸ has the meaning as described herein.

In some embodiments, R⁸ is H, D, —COOH, —C(═O)NH₂, —C(═O)NHCH₃, —C (═O)N(CH₃)₂, —C(═O)—(C₁-C₄ alkyl), —C(═O)—(C₃-C₆ cycloalkyl), —C(═O)-(3-6 membered heterocyclyl), —C(═O)—(C₆-C₁₀ aryl), —C(═O)-(5-10 membered heteroaryl), —C(═O)—(C₁-C₄ alkoxy), —S(═O)₂—(C₁-C₄ alkyl), —S(═O)₂—(C₃-C₆ cycloalkyl), —S(═O)₂-(3-6 membered heterocyclyl), —S(═O)₂—(C₆-C₁₀ aryl), —S(═O)₂-(5-10 membered heteroaryl), C₁-C₄ alkyl, C₂-C₄ alkenyl, C₂-C₄ alkynyl, C₁-C₄ haloalkyl, hydroxy substituted C₁-C₄ alkyl, C₃-C₆ cycloalkyl, 3-6-membered heterocyclyl, C₆-C₁₀ aryl or 5-10 membered heteroaryl.

In other embodiments, R⁸ is H, D, —COOH, —C(═O)NH₂, —C(═O)NHCH₃, —C(═O)N(CH₃)₂, —C(═O)—CH₃, —C(═O)—CH₂CH₃,

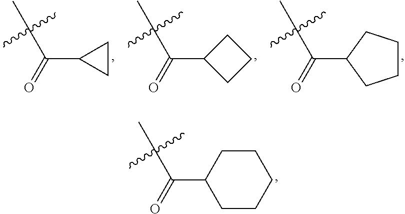 [SMILES: *C(=O)C1CC1 ; *C(=O)C1CCC1 ; *C(=O)C1CCCC1 ; *C(=O)C1CCCCC1]

—C(═O)-(3-6 membered heterocyclyl), —C(═O)-Ph, —C(═O)-(5-6 membered heteroaryl), —C(═O)—OCH₃, —S(═O)₂—CH₃, —S(═O)₂—CH₂CH₃, —S(═O)₂—(C₃-C₆ cycloalkyl), —S(═O)₂-(3-6 membered heterocyclyl), —S(═O)₂-Ph, —S(═O)₂-(5-6 membered heteroaryl), methyl, ethyl, n-propyl, isopropyl, allyl, propenyl, propargyl, propynyl, —CHF₂, —CF₃, —CHFCH₂F, —CF₂CHF₂, —CH₂CHF₂, —CH₂CF₃, —CH₂CF₂CHF₂, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazoly, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl.

In some embodiments, each of R¹, R², and R³ is independently H, D, —COOH, —C (═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, hydroxy substituted C₁-C₄ alkyl, C₃-C₆ cycloalkyl, 3-6 membered heterocyclyl, C₆-C₁₀ aryl or 5-10 membered heteroaryl;

each of R⁴, R⁵ and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxyl substituted C₁-C₄ alkyl, C₃-C₆ cycloalkyl, 3-6 membered heterocyclyl, C₆-C₁₀ aryl or 5-10 membered heteroaryl.

In other embodiments, each of R¹, R², and R³ is independently H, D, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl;

each of R⁴, R⁵ and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl.

In some embodiments, R⁶ is —O—R⁰, R⁷ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxyl substituted C₁-C₄ alkyl, C₃-C₆ cycloalkyl, 3-6 membered heterocyclyl, C₆-C₁₀ aryl, 5-10 membered heteroaryl or —O—R⁰; or

R⁶ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxy substituted C₁-C₄ alkyl, C₃-C₆ cycloalkyl, 3-6 membered heterocyclyl, C₆-C₁₀ aryl or 5-10 membered heteroaryl, R⁷ is —O—R⁰; wherein R⁰ has the meaning as described herein.

In other embodiments, R⁶ is —O—R⁰, R⁷ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl or —O—R⁰; or

R⁶ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl, R⁷ is —O—R⁰; wherein R⁰ has the meaning as described herein.

In other aspect, provided herein is a pharmaceutical composition comprising the compound of formula (I) disclosed in the present invention.

In some embodiments, the pharmaceutical composition disclosed herein further comprises a pharmaceutically acceptable excipient, a carrier, an adjuvant or any combination thereof.

In some embodiments, the pharmaceutical composition according to the present invention further comprises an additional therapeutic agent, wherein the additional therapeutic agent is monoamine oxidase type B inhibitor, dopamine agonist, anticholinergics, glutamate antagonist, levodopa or any combination thereof.

In other aspect, the present invention relates to use of the compound represented by formula (I) or the pharmaceutical composition thereof in the manufacture of a medicament for preventing, treating or lessening an adenosine A_{2A} receptor-related disease in a patient.

In some embodiments, the adenosine A_{2A} receptor-related disease is Parkinson's disease, tumor, pain, depression, dementia, stroke, myocardial ischemia, asthma, alcohol withdrawal, dyskinesia syndrome, restless leg syndrome, dystonia, systemic stiffness, neurodegenerative disorders or osteoporosis.

In other aspect, the present invention relates to use of the compound represented by formula (I) or the pharmaceutical composition thereof in the manufacture of a medicament for antagonizing the adenosine A_{2A} receptor.

In other aspect, the present invention relates to the compound represented by formula (I) or the pharmaceutical composition thereof for use in preventing, treating or lessening an adenosine A_{2A} receptor-related disease in a subject.

In some embodiments, the adenosine A_{2A} receptor-related disease is Parkinson's disease, tumor, pain, depression, dementia, stroke, myocardial ischemia, asthma, alcohol withdrawal, dyskinesia syndrome, restless leg syndrome, dystonia, systemic stiffness, neurodegenerative disorders or osteoporosis.

In other aspect, the present invention relates to the compound represented by formula (I) or the pharmaceutical composition thereof for use in antagonizing adenosine A_{2A} receptor.

In other aspect, the present invention relates to a method of preventing, treating or lessening an adenosine A_{2A} receptor-related disease, which comprises administering a therapeutically effective dose of the compound represented by formula (I) or the pharmaceutical composition thereof.

In some embodiments, the adenosine A_{2A} receptor-related disease is Parkinson's disease, tumor, pain, depression, dementia, stroke, myocardial ischemia, asthma, alcohol withdrawal, dyskinesia syndrome, restless leg syndrome, dystonia, systemic stiffness, neurodegenerative disorders or osteoporosis.

In other aspect, the present invention relates to a method of antagonizing adenosine A_{2A} receptor, which comprises administering a therapeutically effective dose of the compound represented by formula (I) or the pharmaceutical composition thereof.

In other aspect, provided herein is a method of preparing, separating or purifying the compound of formula (I).

Biological test results show that the compound of the present invention can antagonize the adenosine A_{2A} receptor and can be used as a better selective adenosine A_{2A} receptor antagonist.

Any embodiment disclosed herein can be combined with other embodiments as long as they are not contradictory to one another, even though the embodiments are described under different aspects of the invention. In addition, any technical feature in one embodiment can be applied to the corresponding technical feature in other embodiments as long as they are not contradictory to one another, even though the embodiments are described under different aspects of the invention.

The foregoing merely summarizes certain aspects disclosed herein and is not intended to be limiting in nature. These aspects and other aspects and embodiments are described more fully below. All references of this specification are incorporated herein by reference in their entirety. In the event that one or more of the incorporated literature, patents, and similar materials differs from or contradicts this application, this application controls.

### DETAILED DESCRIPTION OF THE INVENTION

### Definitions and General Terminology

Reference will now be made in detail to certain embodiments of the invention, examples of which are illustrated in the accompanying structures and formulas. The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

As used herein, the following definitions shall apply unless otherwise indicated. For purposes of this invention, the chemical elements are identified in accordance with the Periodic Table of the Elements, CAS version, and the Handbook of Chemistry and Physics, 75th Ed. 1994. Additionally, general principles of organic chemistry are described in “Organic Chemistry”, Thomas Sorrell, University Science Books, Sausalito: 1999, and “March's Advanced Organic Chemistry” by Michael B. Smith and Jerry March, John Wiley & Sons, New York: 2007, the entire contents of which are hereby incorporated by reference.

The grammatical articles “a”, “an” and “the”, as used herein, are intended to include “at least one” or “one or more” unless otherwise indicated herein or clearly contradicted by the context. Thus, the articles are used herein to refer to one or more than one (i.e., at least one) of the grammatical objects of the article. By way of example, “a component” means one or more components, and thus, possibly, more than one component is contemplated and may be employed or used in an implementation of the described embodiments.

“Stereoisomers” refers to compounds which have identical chemical constitution, but differ with regard to the arrangement of the atoms or groups in space. Stereoisomers include enantiomer, diastereomers, conformer (rotamer), geometric (cis/trans) isomer, atropisomer, etc.

“Chiral” refers to molecules which have the property of non-superimposability of the mirror image partner, while the term “achiral” refers to molecules which are superimposable on their mirror image partner.

“Enantiomers” refers to two stereoisomers of a compound which are non-superimposable mirror images of one another.

“Racemate” or “racemic mixture” refers to an equimolar mixture of two enantiomers lacking optical activity.

“Diastereomer” refers to a stereoisomer with two or more centers of chirality and whose molecules are not mirror images of one another. Diastereomers have different physical properties, e.g. melting points, boling points, spectral properties and reactivity. A mixture of diastereomers may be separated under high resolution analytical procedures such as electrophoresis and chromatography such as HPLC.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994, all of which are incorporated herein by reference. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. A specific stereoisomer may be referred to as an enantiomer, and a mixture of such stereoisomers is called an enantiomeric mixture. A mixture of enantiomers with a ratio of 50:50 is referred to as a racemic mixture or a racemate, which may occur where there has been no stereoselection or stereospecificity in a chemical reaction or process.

Any asymmetric atom (e.g., carbon or the like) of the compound(s) disclosed herein can be present in racemic or enantiomerically enriched, for example the (R)-, (S)- or (R, S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration.

Depending on the choice of the starting materials and procedures, the compounds can be present in the form of one of the possible stereoisomers or as mixtures thereof, such as racemates and diastereoisomer mixtures, depending on the number of asymmetric carbon atoms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration.

Any resulting mixtures of stereoisomers can be separated on the basis of the physicochemical differences of the constituents, into the pure or substantially pure geometric isomers, enantiomers, diastereomers, for example, by chromatography and/or fractional crystallization.

Any resulting racemates of final products or intermediates can be resolved into the optical antipodes by methods known to those skilled in the art, e.g., by separation of the diastereomeric salts thereof. Racemic products can also be resolved by chiral chromatography, e.g., high performance liquid chromatography (HPLC) using a chiral adsorbent. Preferred enantiomers can also be prepared by asymmetric syntheses. See, for example, Jacques, et, al., Enantiomers, Racemates and Resolutions (Wiley Interscience, New York, 1981); Principles of Asymmetric Synthesis (2nd Ed. Robert E. Gawley, Jeffrey Aube, Elsevier, Oxford, UK, 2012); Eliel, E. L. Stereochemistry of Carbon Compounds (McGraw-Hill, N Y, 1962); Wilen, S. H. Tables of Resolving Agents and Optical Resolutions p. 268 (E. L. Eliel, Ed., Univ. of Notre Dame Press, Notre Dame, Ind. 1972); Chiral Separation Techniques: A Practical Approach (Subramanian, G. Ed., Wiley-VCH Verlag GmbH & Co. KGaA, Weinheim, Germany, 2007). The term “tautomer” or “tautomeric form” refers to structural isomers of different energies which are interconvertible via a low energy barrier. Where tautomerization is possible (e.g. in solution), a chemical equilibrium of tautomers can be reached. For example, protontautomers (also known as prototropic tautomers) include interconversions via migration of a proton, such as keto-enol and imine-enamine isomerizations.

The term “pharmaceutically acceptable” as used herein, refers to those compounds, materials, compositions, and/or dosage forms which are, within the scope of sound medical judgment, suitable for use in contact with the tissues of patients without excessive toxicity, irritation, allergic response, or other problem or complication commensurate with a reasonable benefit/risk ratio, and are effective for their intended use.

The terms “optionally substituted with . . . ” and “unsubstituted or substituted with” can be used interchangeably, i.e. the structure is unsubstituted or substituted with one or more of the substituents described in the present invention, the substituents disclosed herein include, but are not limited to, D, F, Cl, Br, I, N₃, —CD₃, —CN, —NO₂, —NH₂, —OH, —SH, —COOH, —C(═O)NH₂, —C(═O)NHCH₃, —C(═O)N(CH₃)₂, —C(═O)-(alkyl), —C(═O)-(cycloalkyl), —C(═O)-(heterocyclyl), —C(═O)-(aryl), —C(═O)-(heteroaryl), —C(═O)-(alkoxy), —S(═O)₂-(alkyl), —S(═O)₂-(cycloalkyl), —S(═O)₂-(heterocyclyl), —S(═O)₂-(aryl), —S(═O)₂-(heteroaryl), alkyl, alkenyl, alkynyl, haloalkyl, alkoxy, haloalkoxy, alkylthio, alkylamino, hydroxy-substituted alkyl, cycloalkyl, heterocyclyl, aryl, heteroaryl, and so on.

In general, the term “substituted” refers to the replacement of one or more hydrogen radicals in a given structure or radical with a specified substituent. Unless otherwise indicated, a substituent may substitute at any substitutable position of a radical. When more than one positions of a given structure can be substituted with one or more specified substituents, the substituents may be either the same or different at each position.

Furthermore, what need to be explained is that the phrase “each . . . is independently” and “each of . . . and . . . is independently”, unless otherwise stated, should be broadly understood. The specific options expressed by the same symbol are independent of each other in different groups; or the specific options expressed by the same symbol are independent of each other in same groups.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, “patient” refers to a human (including adults and children) or other animal. In one embodiment, “patient” refers to a human.

The term “comprise” is an open expression, it means comprising the contents disclosed herein, but doesn't exclude other contents.

At various places in the present specification, substituents of compounds disclosed herein are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl.

At various places in the present specification, linking substituents are described. Where the structure clearly requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “D” or “²H” refers to a single deuterium atom.

The terms “halogen” and “halo” can be used interchangeably, which refer to Fluoro (F), Chloro (Cl), Bromo (Br), or Iodo (I).

The term “oxo” refers to the group “═O”, which can be used interchangeably with “carbonyl”.

The term “heteroatom” refers to oxygen, sulfur, nitrogen, phosphorus and silicon, including any oxidized form of nitrogen, sulfur, or phosphorus; primary, secondary, tertiary amines and quaternary ammonium salts forms; or a substitutable nitrogen of a heterocyclic ring, for example, N (as in 3,4-dihydro-2H-pyrrolyl), NH (as in pyrrolidinyl) or NR′ (as in N-substituted pyrrolidinyl, wherein R′ is the substituent described herein).

The term “alkyl” or “alkyl group” refers to a saturated linear or branched-chain monovalent hydrocarbon group of 1-20 carbon atoms, wherein the alkyl group is optionally substituted with one or more substituents described herein. In one embodiment, the alkyl group contains 1-6 carbon atoms. In other embodiment, the alkyl group contains 1-4 carbon atoms. In still other embodiment, the alkyl group contains 1-3 carbon atoms. Examples of alkyl groups include, but are not limited to, methyl (Me, —CH₃), ethyl (Et, —CH₂CH₃), n-propyl (n-Pr, —CH₂CH₂CH₃), isopropyl (i-Pr, —CH(CH₃)₂), n-butyl (n-Bu, —CH₂CH₂CH₂CH₃), isobutyl (i-Bu, —CH₂CH(CH₃)₂), sec-butyl (s-Bu, —CH(CH₃) CH₂CH₃), tert-butyl (t-Bu, —C(CH₃)₃), etc.

The term “alkenyl” refers to linear or branched-chain monovalent hydrocarbon radical of 2 to 12 carbon atoms with at least one site of unsaturation, i.e., a carbon-carbon, sp² double bond, wherein the alkenyl radical may be optionally substituted with one or more substituents described herein, and includes radicals having “cis” and “trans” orientations, or alternatively, “E” and “Z” orientations. In some embodiments, the alkenyl group contains 2 to 8 carbon atoms. In other embodiments, the alkenyl group contains 2 to 6 carbon atoms. In still other embodiments, the alkenyl group contains 2 to 4 carbon atoms. Some non-limiting examples of alkenyl group include vinyl (—CH═CH₂), allyl (—CH₂CH═CH₂), 1-propenyl (i.e, propenyl, —CH═CH—CH₃), and the like.

The term “alkynyl” refers to a linear or branched-chain monovalent hydrocarbon radical of 2 to 12 carbon atoms with at least one site of unsaturation, i.e., a carbon-carbon sp triple bond, wherein the alkynyl radical may be optionally substituted independently with one or more substituents described herein. In some embodiments, the alkynyl group contains 2 to 8 carbon atoms. In other embodiments, the alkynyl group contains 2 to 6 carbon atoms. In still other embodiments, the alkynyl group contains 2 to 4 carbon atoms. Examples of alkynyl groups include, but are not limited to, ethynyl (—C≡CH), propargyl (—CH₂C≡CH), 1-propynyl (i.e, propynyl, —C≡C—CH₃), and the like.

The term “alkoxy” refers to an alkyl group, as previously defined, attached to the parent molecular moiety via an oxygen atom. Unless otherwise specified, the alkoxy group contains 1-12 carbon atoms. In one embodiment, the alkoxy group contains 1-6 carbon atoms. In other embodiment, the alkoxy group contains 1-4 carbon atoms. In still other embodiment, the alkoxy group contains 1-3 carbon atoms. The alkoxy group may be optionally substituted with one or more substituents disclosed herein.

Examples of alkoxy groups include, but are not limited to, methoxy (MeO, —OCH₃), ethoxy (EtO, —OCH₂CH₃), 1-propoxy (n-PrO, n-propoxy, —OCH₂CH₂CH₃), 2-propoxy (i-PrO, i-propoxy, —OCH(CH₃)₂), 1-butoxy (n-BuO, n-butoxy, —OCH₂CH₂CH₂CH₃), 2-methyl-1-propoxy (i-BuO, i-butoxy, —OCH₂CH(CH₃)₂), 2-butoxy (s-BuO, s-butoxy, —OCH(CH₃)CH₂CH₃), 2-methyl-2-propoxy (t-BuO, t-butoxy, —OC(CH₃)₃), etc.

The term “alkylthio” refers to an alkyl group, as previously defined, attached to the parent molecular moiety via a sulfur atom. Unless otherwise specified, the alkylthio group contains 1-12 carbon atoms. In one embodiment, the alkylthio group contains 1-6 carbon atoms. In other embodiment, the alkylthio group contains 1-4 carbon atoms. In still other embodiment, the alkylthio group contains 1-3 carbon atoms. The alkylthio group may be optionally substituted with one or more substituents disclosed herein.

Examples of alkylthio groups include, but are not limited to, methylthio (MeS, —SCH₃), ethylthio (EtS, —SCH₂CH₃), 1-propylthio (n-PrS, n-propylthio, —SCH₂CH₂CH₃), 2-propylthio (i-PrS, i-propylthio, —SCH(CH₃)₂), 1-butylthio (n-BuS, n-butylthio, —SCH₂CH₂CH₂CH₃), 2-methyl-1-propylthio (i-BuS, i-butylthio, —SCH₂CH(CH₃)₂), 2-butylthio (s-BuS, s-butylthio, —SCH(CH₃)CH₂CH₃), 2-methyl-2-propylthio (t-BuS, t-butylthio, —SC(CH₃)₃), etc.

The term “alkylamino” embraces “N-alkylamino” and “N,N-dialkylamino”, wherein an amino group is independently substituted with one or two alkyl radicals and wherein the alkyl group is as defined herein. Suitable alkylamino radical may be monoalkylamino or dialkylamino. Examples of the alkylamino radical include, but are not limited to, N-methylamino (methylamino), N-ethylamino (ethylamino), N,N-dimethylamino (dimethylamino), N,N-diethylamino (diethylamino), and the like. And wherein the alkylamino radical is optionally substituted with one or more substituents described herein.

The term “hydroxy-substituted alkyl” refers to an alkyl group substituted with one or more hydroxy groups, wherein the alkyl is as defined herein. Examples of such group include, but are not limited to, hydroxymethyl, 2-hydroxyethyl, 2-hydroxy-1-propyl, 3-hydroxy-1-propyl, 2,3-dihydroxypropyl, and the like.

The term “haloalkyl” means that an alkyl group is substituted with one or more halogen atoms, wherein the alkyl group is as defined herein. Examples of such group include, but are not limited to, —CHF₂, —CF₃, —CHFCH₂F, —CF₂CHF₂, —CH₂CHF₂, —CH₂CF₃, —CHFCH₃, —CH₂CH₂F, —CF₂CH₃, —CH₂CF₂CHF₂, etc. In one embodiment, the C₁-C₆ haloalkyl group includes a fluorine-substituted C₁-C₆ alkyl group; in another embodiment, the C₁-C₄ haloalkyl group includes a fluorine-substituted C₁-C₄ alkyl group; in another embodiment, the C₁-C₂ haloalkyl group includes a fluorine-substituted C₁-C₂ alkyl group.

The term “haloalkoxy” means that an alkoxy group is substituted with one or more halogen atoms, wherein the alkoxy group is as defined herein. Examples of such group include, but are not limited to, —OCHF₂, —OCF₃, —OCHFCH₂F, —OCF₂CHF₂, —OCH₂CHF₂, —OCH₂CF₃, —OCHFCH₃, —OCH₂CH₂F, —OCF₂CH₃, —OCH₂CF₂CHF₂, etc. In one embodiment, the C₁-C₆ haloalkoxy group includes a fluorine-substituted C₁-C₆ alkoxy group; in another embodiment, the C₁-C₄ haloalkoxy group includes a fluorine-substituted C₁-C₄ alkoxy group; in another embodiment, the C₁-C₂ haloalkoxy group includes a fluorine-substituted C₁-C₂ alkoxy group.

The term “consisting of n atoms” or “n-membered” are used interchangeably herein, where n is an integer, which typically describes the number of ring-forming atoms in a moiety, and the number of ring-forming atoms is n. For example, a 5-10 membered heteroaryl group means a heteroaryl group composed of 5, 6, 7, 8, 9 or 10 ring atoms. As another example, piperidinyl is a heterocyclyl consisting of 6 ring atoms or a 6 membered heterocyclyl, and pyridyl is a heteroaryl consisting of 6 ring atoms or a 6 membered heteroaryl.

The term “cycloalkyl” refers to a monovalent or multivalent saturated ring having 3 to 12 carbon atoms as a monocyclic, bicyclic, or tricyclic ring system. And wherein the bicyclic or tricyclic ring system may include fused ring, bridged ring and spiro ring. In some embodiments, the cycloalkyl group contains 3 to 10 carbon atoms. In other embodiments, the cycloalkyl group contains 3 to 8 carbon atoms. In still other embodiments, the cycloalkyl group contains 3 to 6 carbon atoms. The cycloalkyl radical is optionally substituted with one or more substituents described herein. Examples of cycloalkyl groups include, but are not limited to, cyclopropyl, cyclobutyl, cyclopentyl, cyclohexyl and the like.

The term “heterocycle”, “heterocyclyl”, or “heterocyclic ring” as used interchangeably herein refers to a saturated or partially unsaturated and nonaromatic monocyclic, bicyclic or tricyclic ring system containing 3-12 ring atoms, wherein the bicyclic or tricyclic ring system may include a fused ring, bridged ring and spiro ring. Wherein one or more atoms on the ring each are independently replaced by heteroatom, the heteroatom is as defined herein. Of which a —CH₂— group can optionally be replaced by a —C(═O)— group, ring sulfur atoms may be optionally oxidized to form S-oxides, and ring nitrogen atoms may be optionally oxidized to form N-oxides. In some embodiments, the heterocyclyl group is monocyclic heterocyclyl having 3-8 ring members (e.g., 2 to 6 carbon atoms and 1 to 3 heteroatoms selected from N, O, P and S, wherein the S or P is optionally substituted with one or more oxygen atoms to give the groups like SO or SO₂, PO or PO₂); In other embodiments, the heterocyclyl group is monocyclic heterocyclyl having 3-6 ring members (e.g., 2 to 5 carbon atoms and 1 to 3 heteroatoms selected from N, O, P and S, wherein the S or P is optionally substituted with one or more oxygen atoms to give the groups like SO or SO₂, PO or PO₂); In another embodiments, the heterocyclyl group is bicyclic heterocyclyl having 7-12 ring members (e.g., 4 to 9 carbon atoms and 1 to 3 heteroatoms selected from N, O, P and S, wherein the S or P is optionally substituted with one or more oxygen atoms to give the groups like SO or SO₂, PO or PO₂); The heterocyclyl group is optionally substituted with one or more substituents described herein.

The ring atom of the heterocyclic group may be a carbon atom radical or heteroatom radical. Of which a —CH₂— group can optionally be replaced by a —C(═O)— group, ring sulfur atoms may be optionally oxidized to form S-oxides, and ring nitrogen atoms may be optionally oxidized to form N-oxides. Some non-limiting examples of heterocyclyl group include oxiranyl, azetidinyl, oxetanyl, thietanyl, pyrrolidinyl, 2-pyrrolinyl, 3-pyrrolinyl, pyrazolinyl, pyrazolidinyl, imidazolinyl, imidazolidinyl, tetrahydrofuranyl, dihydrofuranyl, tetrahydrothienyl, dihydrothienyl, 1,3-dioxolanyl, dithiolanyl, tetrahydropyranyl, dihydropyranyl, 2H-pyranyl, 4H-pyranyl, tetrahydrothiopyranyl, piperidinyl, morpholinyl, thiomorpholinyl, piperazinyl, dioxanyl, dithianyl, thioxanyl, homopiperazinyl, homopiperidinyl, oxepanyl, thiepanyl, oxazepinyl, diazepinyl, thiazepinyl, 2-oxa-5-azabicyclo[2.2.1]hept-5-yl, and the like. Some non-limiting examples of heterocyclyl wherein —CH₂— group is replaced by —C(═O)— moiety include 2-oxopyrrolidinyl, oxo-1,3-thiazolidinyl, 2-piperidinonyl, 3,5-dioxopiperidinyl, pyrimidinedionyl, and the like. Some non-limiting examples of oxidized ring sulfur atoms of the heterocyclyl group include sulfolanyl, 1,1-dioxo-thiomorpholinyl, and the like. The heterocyclyl group is optionally substituted with one or more substituents described herein.

The term “aryl” refers to monocyclic, bicyclic and tricyclic carbocyclic ring systems having a total of six to fourteen ring members, or six to twelve ring members, or six to ten ring members, wherein at least one ring in the system is aromatic, wherein each ring in the system contains 3 to 7 ring members. The aryl group is generally, but not necessarily bonded to the parent molecule through an aromatic ring of the aryl group. The term “aryl” and “aromatic ring” can be used interchangeably herein. Examples of the aryl group would include phenyl, indenyl, naphthyl, and anthracene. The aryl radical is optionally substituted with one or more substituents described herein.

The term “heteroaryl” refers to monocyclic, bicyclic and tricyclic ring systems having a total of five to twelve ring members, or five to ten ring members, or five to six ring members, wherein at least one ring in the system is aromatic, and in which at least one ring system contains one or more heteroatoms, and wherein each ring system contains a 5 to 7 members ring. The heteroaryl group is generally, but not necessarily bonded to the parent molecule through an aromatic ring of the heteroaryl group. The term “heteroaryl” may be used interchangeably with the term “heteroaryl ring”, “aromatic heterocyclic” or the term “heteroaromatic compound”. The heteroaryl group is optionally substituted with one or more substituents disclosed herein. In one embodiment, a 5-10 membered heteroaryl comprises 1, 2, 3 or 4 heteroatoms independently selected from O, S and N.

Some non-limiting examples of heteroaryl rings include 2-furanyl, 3-furanyl, N-imidazolyl, 2-imidazolyl, 4-imidazolyl, 5-imidazolyl, 3-isoxazolyl, 4-isoxazolyl, 5-isoxazolyl, 2-oxazolyl, 4-oxazolyl, 5-oxazolyl, N-pyrrolyl, 2-pyrrolyl, 3-pyrrolyl, 2-pyridyl, 3-pyridyl, 4-pyridyl, 2-pyrimidinyl, 4-pyrimidinyl, 5-pyrimidinyl, pyridazinyl (e.g., 3-pyridazinyl), 2-thiazolyl, 4-thiazolyl, 5-thiazolyl, tetrazolyl (e.g., 5-tetrazolyl), triazolyl (e.g., 2-triazolyl and 5-triazolyl), 2-thienyl, 3-thienyl, pyrazolyl (e.g., 2-pyrazolyl), isothiazolyl, 1,2,3-oxadiazolyl, 1,2,5-oxadiazolyl, 1,2,4-oxadiazolyl, 1,2,3-triazolyl, 1,2,3-thiadiazolyl, 1,3,4-thiadiazolyl, 1,2,5-thiadiazolyl, pyrazinyl, 1,3,5-triazinyl, and the following bicycles: benzimidazolyl, benzofuryl, benzothiophenyl, indolyl (e.g., 2-indolyl), purinyl, quinolinyl (e.g., 2-quinolinyl, 3-quinolinyl, 4-quinolinyl), and isoquinolinyl (e.g., 1-isoquinolinyl, 3-isoquinolinyl or 4-isoquinolinyl), imidazo[1,2-a]pyridyl, pyrazolo[1,5-a]pyridyl, pyrazolo[1,5-a]pyrimidyl, imidazo[1,2-b]pyridazinyl, [1,2,4]triazolo[4,3-b]pyridazinyl, [1,2,4]triazolo[1,5-a]pyrimidinyl, or [1,2,4]triazolo[1,5-a]pyridyl, etc.

The term “protecting group” or “PG” refers to a substituent that is commonly employed to block or protect a particular functionality while reacting with other functional groups on the compound. For example, an “amino-protecting group” is a substituent attached to an amino group that blocks or protects the amino functionality in the compound. Suitable amino-protecting groups include acetyl, trifluoroacetyl, t-butoxy-carbonyl (BOC, Boc), benzyloxycarbonyl (CBZ, Cbz) and 9-fluorenylmethylenoxy-carbonyl (Fmoc). Similarly, a “hydroxy-protecting group” refers to a substituent of a hydroxy group that blocks or protects the hydroxy functionality. Some non-limiting examples of suitable hydroxy-protecting groups include trialkylsilyl, acetyl, benzoyl, and benzyl. A “carboxy-protecting group” refers to a substituent of the carboxy group that blocks or protects the carboxy functionality. Common carboxy-protecting groups include —CH₂CH₂SO₂Ph, cyanoethyl, 2-(trimethylsilyl)ethyl, 2-(trimethylsilyl) ethoxy-methyl, 2-(p-toluenesulfonyl) ethyl, 2-(p-nitrophenylsulfonyl)ethyl, 2-(diphenylphosphino)ethyl, nitroethyl and the like. For a general description of protecting groups and their use, see Greene et, al., Protective Groups in Organic Synthesis, John Wiley & Sons, New York, 1991 and Kocienski et, al., Protecting Groups, Thieme, Stuttgart, 2005.

The term “prodrug” refers to a compound that is transformed in vivo into a compound of formula (I) or formula (II). Such a transformation can be affected, for example, by hydrolysis of the prodrug form in blood or enzymatic transformation to the parent form in blood or tissue. Prodrugs of the compounds disclosed herein may be, for example, esters. Some common esters which have been utilized as prodrugs are phenyl esters, aliphatic (C₁₋₂₄) esters, acyloxymethyl esters, carbonates, carbamates and amino acid esters. For example, a compound disclosed herein that contains a hydroxy group may be acylated at this position in its prodrug form. Other prodrug forms include phosphates, such as, those phosphate compounds derived from the phosphonation of a hydroxy group on the parent compound.

A “metabolite” is a product produced through metabolism in the body of a specified compound or salt thereof. The metabolites of a compound may be identified using routine techniques known in the art and their activities determined using tests such as those described herein. Such products may result for example from oxidation, reduction, hydrolysis, amidation, deamidation, esterification, deesterification, enzyme cleavage, and the like, of the administered compound. Accordingly, the invention includes metabolites of compounds disclosed herein, including metabolites produced by contacting a compound disclosed herein with a mammal for a sufficient time period.

A “pharmaceutically acceptable salts” refers to organic or inorganic salts of a compound disclosed herein. Pharmaceutically acceptable salts are well known in the art. For example, S. M. Berge et, al., describe pharmaceutically acceptable salts in detail in J. Pharmaceutical Sciences, 1977, 66: 1-19, which is incorporated herein by reference. Some non-limiting examples of pharmaceutically acceptable and nontoxic salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid and malonic acid or by using other methods used in the art such as ion exchange. Other pharmaceutically acceptable salts include adipate, alginate, ascorbate, aspartate, benzenesulfonate, benzoate, bisulfate, borate, butyrate, camphanic acid salt, camphorsulfonate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, laurylsulfate, malate, malonate, methanesulfonate, 2-naphthalenesulfonate, nicotinate, nitrate, oleate, palmitate, pamoate, pectinate, persulfate, 3-phenylpropionate, picrate, pivalate, propionate, stearate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. This invention also envisions the quaternization of any basic nitrogen-containing groups of the compounds disclosed herein. Water or oil soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, C₁-C₈ sulfonate or aryl sulfonate.

The term “solvate” refers to an association or complex of one or more solvent molecules and a compound disclosed herein. Some non-limiting examples of the solvent that form solvates include water, isopropanol, ethanol, methanol, dimethylsulfoxide (DMSO), ethyl acetate, acetic acid, ethanolamine or a combination thereof. The term “hydrate” refers to the complex where the solvent molecule is water.

The term “hydrate” can be used when said solvent is water. In one embodiment, one water molecule is associated with one molecule of the compounds disclosed herein, such as a hydrate. In another embodiment, more than one water molecule may be associated with one molecule of the compounds disclosed herein, such as a dihydrate. In still another embodiment, less than one water molecule may be associated with one molecule of the compounds disclosed herein, such as a hemihydrate. Furthermore, all the hydrates of the invention retain the biological effectiveness of the non-hydrate form of the compounds disclosed herein.

The term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

The term “preventing” or “prevention” refers to a reduction in risk of acquiring a disease or disorder (i.e., causing at least one of the clinical symptoms of the disease not to develop in a subject that may be exposed to or predisposed to the disease but does not yet experience or display symptoms of the disease).

Unless otherwise stated, all suitable isotopic variations, stereoisomers, tautomers, solvates, metabolites, salts and pharmaceutically acceptable prodrugs of the compounds disclosed herein are within the scope of the invention.

All stereisomers of the structure disclosed herein are considered within the scope of the invention whether the stereochemistry of the structure is indicated or not, and which are interpreted as disclosed compounds of the invention and included in the invention. When the stereochemistry of a structure is indicated by solid wedge or dash line, the stereisomer of the structure is definite.

N-oxides of the compound disclosed herein are also included in the invention. N-oxides of the compound of the invention can be prepared by oxidizing corresponding nitrogen-containing alkaline substances with common oxidants (such as hydrogen peroxide) under a rising temperature in the presence of an acid, such as acetic acid, or by reacting with peracid in a suitable solvent, e.g., by reacting with peracetic acid in dichloromethane, ethyl acetate or methyl acetate, or by reacting with 3-chloroperoxybenzoic acid in chloroform or dichloromethane.

The compound of formula (I) or (II) can exist in salt forms. In one embodiment, the salt is a pharmaceutically acceptable salt thereof. The term “pharmaceutically acceptable” refers to that the substance or composition must be chemically and/or toxicologically compatible with the other ingredients comprising a formulation, and/or the mammal being treated therewith. In another embodiment, the salt may not be a pharmaceutically acceptable salt, may be an intermediate used for preparing and/or purifying the compound of formula (I) or (II) and/or isolating an enantiomer from the compound of formula (I) or (II).

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent isotopically unenriched forms as well as isotopically enriched forms of the compounds. Isotopically enriched compounds have the structure depicted by the general formula given herein, except that one or more atoms are replaced by atom(s) having a selected atomic mass or mass number. Exemplary isotopes that can be introduced into the compounds of the present invention include isotopes of hydrogen, carbon, nitrogen, oxygen, phosphorus, sulfur, fluorine, and chlorine, such as ²H, ³H, ¹¹C, ¹³C, ¹⁴C, ¹⁵N, ¹⁷O, ¹⁸O, ¹⁸F, ³¹P, ³²P, ³⁵S, ³⁶Cl and ¹²⁵I.

In other aspect, provided herein is an intermediate for preparing the compound of formula (I) or (II).

In other aspect, provided herein is a pharmaceutical composition comprising the compound disclosed herein. In some embodiments, the pharmaceutical composition disclosed herein further comprises at least one of pharmaceutically acceptable carrier, excipient, adjuvant, solvent or a combination thereof. In other embodiment, the pharmaceutical composition can be liquid, solid, semi-solid, gel or spray.

### DESCRIPTION OF COMPOUNDS OF THE INVENTION

The 8-substituted styrylxanthine derivatives, pharmaceutically acceptable salts, pharmaceutical formulations and compositions thereof disclosed herein can be used to antagonize adenosine A_{2A} receptors, and have potential therapeutic use for adenosine A_{2A} receptor-related diseases, especially PD (Parkinson's Disease). The present invention further describes the synthetic method of the compound. The compounds of the invention show good bioactivity.

In one aspect, provided herein is a compound having Formula (I) or a stereoisomer, a tautomer, an N-oxide, a hydrate, a solvate, a metabolite, a pharmaceutically acceptable salt or a prodrug thereof,

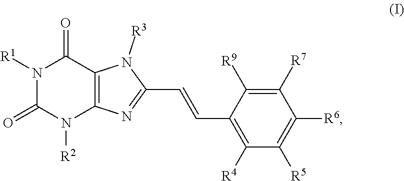

wherein, each of R¹, R², R³, R⁴, R⁵, R⁶, R⁷ and R⁹ is as defined herein.

In some embodiments, each of R¹, R², and R³ is independently H, D, —COOH, —C (═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, hydroxy substituted C₁-C₆ alkyl, C₃-C₈ cycloalkyl, 3-8 membered heterocyclyl, C₆-C₁₀ aryl or 5-10 membered heteroaryl.

In some embodiments, each of R⁴, R⁵, and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxy substituted C₁-C₆ alkyl, C₃-C₈ cycloalkyl, 3-8 membered heterocyclyl, C₆-C₁₀ aryl or 5-10 membered heteroaryl.

In some embodiments, R⁶ is —O—R⁰, R⁷ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxyl substituted C₁-C₆ alkyl, C₃-C₈ cycloalkyl, 3-8 membered heterocyclyl, C₆-C₁₀ aryl, 5-10 membered heteroaryl or —O—R⁰; or

R⁶ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxy substituted C₁-C₆ alkyl, C₃-C₈ cycloalkyl, 3-8 membered heterocyclyl, C₆-C₁₀ aryl or 5-10 membered heteroaryl, R⁷ is —O—R⁰; wherein R⁰ has the meaning as described herein.

In some embodiments, R⁰ is

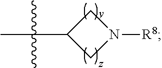

wherein y, z and R⁸ have the meaning as described herein.

In some embodiments, y is 1, 2 or 3.

In some embodiments, z is 1, 2 or 3.

In some embodiments, R⁸ is H, D, —COOH, —C(═O)NH₂, —C(═O)NHCH₃, —C(═O)N(CH₃)₂, —C(═O)—(C₁-C₆ alkyl), —C(═O)—(C₃-C₈ cycloalkyl), —C(═O)-(3-8 membered heterocyclyl), —C(═O)—(C₆-C₁₀ aryl), —C(═O)-(5-10 membered heteroaryl), —C(═O)—(C₁-C₆ alkoxy), —S(═O)₂—(C₁-C₆ alkyl), —S(═O)₂—(C₃-C₈ cycloalkyl), —S(═O)₂-(3-8 membered heterocyclyl), —S(═O)₂—(C₆-C₁₀ aryl), —S(═O)₂-(5-10 membered heteroaryl), C₁-C₆ alkyl, C₂-C₆ alkenyl, C₂—C alkynyl, C₁-C₆ haloalkyl, hydroxy substituted C₁-C₆ alkyl, C₃-C₈ cycloalkyl, 3-8 membered heterocyclyl, C₆-C₁₀ aryl or 5-10 membered heteroaryl.

In some embodiments, R⁰ is

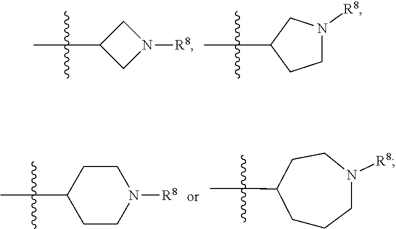

wherein R⁸ has the meaning as described herein.

In some embodiments, R⁰ is

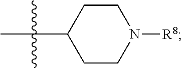

wherein R⁸ has the meaning as described herein.

In some embodiments, R⁸ is H, D, —COOH, —C(═O)NH₂, —C(═O)NHCH₃, —C(═O)N(CH₃)₂, —C(═O)—(C₁-C₄ alkyl), —C(═O)—(C₃-C₆ cycloalkyl), —C(═O)-(3-6 membered heterocyclyl), —C(═O)—(C₆-C₁₀ aryl), —C(═O)-(5-10 membered heteroaryl), —C(═O)—(C₁-C₄ alkoxy), —S(═O)₂—(C₁-C₄ alkyl), —S(═O)₂—(C₃-C₆ cycloalkyl), —S(═O)₂-(3-6 membered heterocyclyl), —S(═O)₂—(C₆-C₁₀ aryl), —S(═O)₂-(5-10 membered heteroaryl), C₁-C₄ alkyl, C₂-C₄ alkenyl, C₂-C₄ alkynyl, C₁-C₄ haloalkyl, hydroxy substituted C₁-C₄ alkyl, C₃-C₆ cycloalkyl, 3-6 membered heterocyclyl, C₆-C₁₀ aryl or 5-10 membered heteroaryl.

In other embodiments, R⁸ is H, D, —COOH, —C(═O)NH₂, —C(═O)NHCH₃, —C(═O)N(CH₃)₂, —C(═O)—CH₃, —C(═O)—CH₂CH₃,

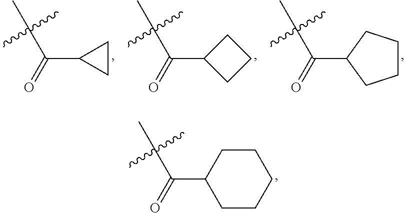 [SMILES: *C(=O)C1CC1 ; *C(=O)C1CCC1 ; *C(=O)C1CCCC1 ; *C(=O)C1CCCCC1]

—C(═O)-(3-6 membered heterocyclyl), —C(═O)-Ph, —C(═O)-(5-6 membered heteroaryl), —C(═O)—OCH₃, —S(═O)₂—CH₃, —S(═O)₂—CH₂CH₃, —S(═O)₂—(C₃-C₆ cycloalkyl), —S(═O)₂-(3-6 membered heterocyclyl), —S(═O)₂-Ph, —S(═O)₂-(5-6 membered heteroaryl), methyl, ethyl, n-propyl, isopropyl, allyl, propenyl, propargyl, propynyl, —CHF₂, —CF₃, —CHFCH₂F, —CF₂CHF₂, —CH₂CHF₂, —CH₂CF₃, —CH₂CF₂CHF₂, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazoly, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl.

In some embodiments, each of R¹, R², and R³ is independently H, D, —COOH, —C (═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, hydroxy substituted C₁-C₄ alkyl, C₃-C₆ cycloalkyl, 3-6 membered heterocyclyl, C₆-C₁₀ aryl or 5-10 membered heteroaryl.

In other embodiments, each of R¹, R², and R³ is independently H, D, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl.

In some embodiments, each of R⁴, R⁵, and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxy substituted C₁-C₄ alkyl, C₃-C₆ cycloalkyl, 3-6 membered heterocyclyl, C₆-C₁₀ aryl or 5-10 membered heteroaryl.

In other embodiments, each of R⁴, R⁵, and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl.

In some embodiments, R⁶ is —O—R⁰, R⁷ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxyl substituted C₁-C₄ alkyl, C₃-C₆ cycloalkyl, 3-6 membered heterocyclyl, C₆-C₁₀ aryl, 5-10 membered heteroaryl or —O—R⁰; or

R⁶ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxy substituted C₁-C₄ alkyl, C₃-C₆ cycloalkyl, 3-6 membered heterocyclyl, C₆-C₁₀ aryl or 5-10 membered heteroaryl, R⁷ is —O—R⁰; wherein R⁰ has the meaning as described herein.

In other embodiments, R⁶ is —O—R⁰, R⁷ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl or —O—R⁰; or

R⁶ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl, R⁷ is —O—R⁰; wherein R⁰ has the meaning as described herein.

In some embodiments, provided herein is a compound having formula (II) or a stereoisomer, a tautomer, an N-oxide, a hydrate, a solvate, a metabolite, a pharmaceutically acceptable salt or a prodrug thereof,

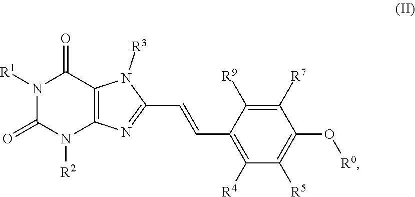

wherein, each of R¹, R², R³, R⁴, R⁵, R⁷, R⁹ and R⁰ is as defined herein.

In some embodiments, the compound disclosed herein has one of the following structures or a stereoisomer, a tautomer, an N-oxide, a hydrate, a solvate, a metabolite, a pharmaceutically acceptable salt or a prodrug thereof, but is in no way limited to:

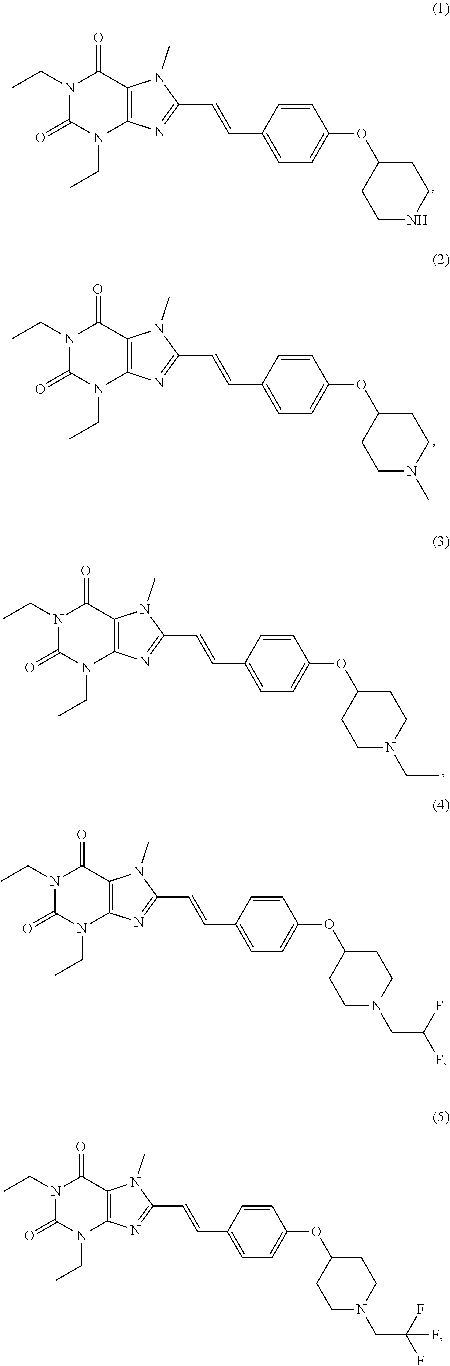

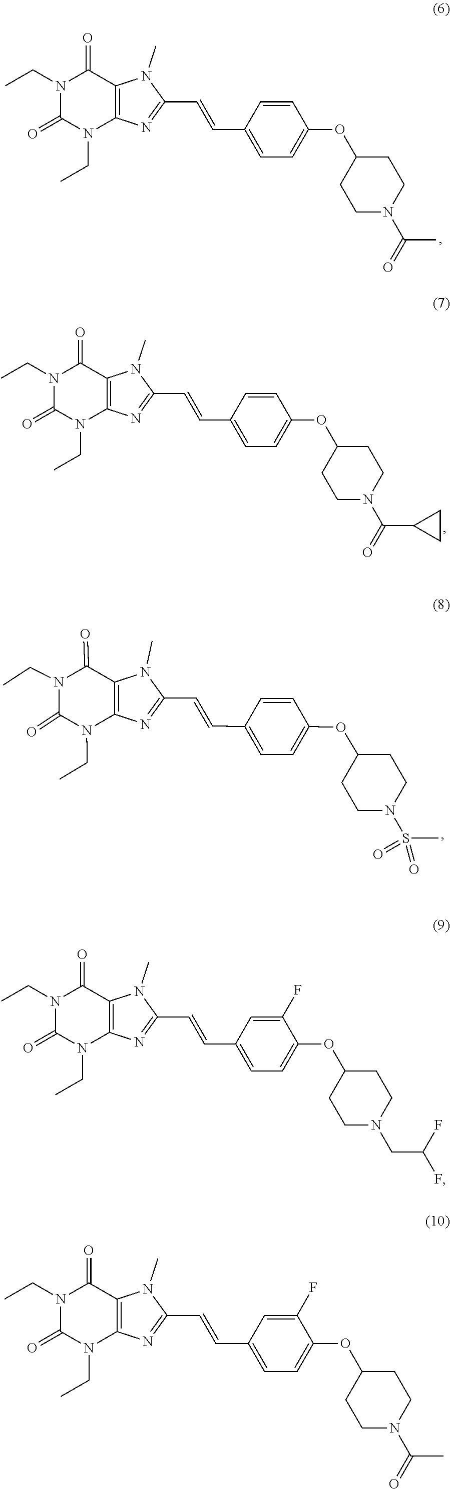

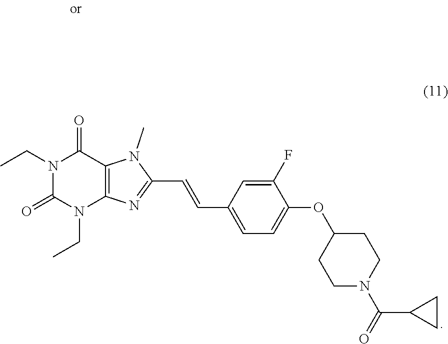 [SMILES: CCn1c(=O)c2c(nc(/C=C/c3ccc(OC4CCN(C(=O)C5CC5)CC4)c(F)c3)n2C)n(CC)c1=O]

In other aspect, provided herein is a pharmaceutical composition comprising the compound of formula (I) or (II) disclosed in the present invention.

In some embodiments, the pharmaceutical composition disclosed herein further comprises a pharmaceutically acceptable excipient, a carrier, an adjuvant or a combination thereof.

In other embodiment, the pharmaceutical composition according to the present invention further comprises an additional therapeutic agent, wherein the additional therapeutic agent is monoamine oxidase type B inhibitors such as selegiline and rasagiline, dopamine agonists such as bromocriptine, cabergoline, pergolide, pramipexole, ropinirole and rotigotine, anticholinergic drugs such as trihexphenidyl, benztropine, orfenadrine and procyclidine, glutamate antagonists such as amantadine, levodopa (optionally in combination with a carboxylase inhibitor such as carbidopa and benzylhydrazine, a COMT inhibitor such as tocapone and entacapone or both a carboxylase inhibitor and a COMT inhibitor) or any combination thereof.

In other aspect, the present invention relates to use of the compound represented by formula (I) or (II) or the pharmaceutical composition thereof in the manufacture of a medicament for preventing, treating or lessening an adenosine A_{2A} receptor-related disease in a patient.

In some embodiments, the adenosine A_{2A} receptor-related disease is Parkinson's disease, tumor, pain, depression, dementia, stroke, myocardial ischemia, asthma, alcohol withdrawal, dyskinesia syndrome, restless leg syndrome, dystonia, systemic stiffness, neurodegenerative disorders or osteoporosis.

In other embodiments, the adenosine A_{2A} receptor-related disease is PD (Parkinson's Disease).

In other aspect, the present invention relates to use of the compound represented by formula (I) or (II) or the pharmaceutical composition thereof in the manufacture of a medicament for antagonizing the adenosine A_{2A} receptor.

In other aspect, the present invention relates to the compound represented by formula (I) or (II) or the pharmaceutical composition thereof for use in preventing, treating or lessening an adenosine A_{2A} receptor-related disease in a subject.

In some embodiments, the adenosine A_{2A} receptor-related disease is Parkinson's disease, tumor, pain, depression, dementia, stroke, myocardial ischemia, asthma, alcohol withdrawal, dyskinesia syndrome, restless leg syndrome, dystonia, systemic stiffness, neurodegenerative disorders or osteoporosis.

In other embodiments, the adenosine A_{2A} receptor-related disease is PD (Parkinson's Disease).

In other aspect, the present invention relates to the compound represented by formula (I) or (II) or the pharmaceutical composition thereof for use in antagonizing adenosine A_{2A} receptor.

In other aspect, the present invention relates to a method of preventing, treating or lessening an adenosine A_{2A} receptor-related disease, which comprises administering a therapeutically effective dose of the compound represented by formula (I) or (II) or the pharmaceutical composition thereof.

In some embodiments, the adenosine A_{2A} receptor-related disease is Parkinson's disease, tumor, pain, depression, dementia, stroke, myocardial ischemia, asthma, alcohol withdrawal, dyskinesia syndrome, restless leg syndrome, dystonia, systemic stiffness, neurodegenerative disorders or osteoporosis.

In other embodiments, the adenosine A_{2A} receptor-related disease is PD (Parkinson's Disease).

In other aspect, the present invention relates to a method of antagonizing adenosine A_{2A} receptor, which comprises administering a therapeutically effective dose of the compound represented by formula (I) or (II) or the pharmaceutical composition thereof.

In other aspect, provided herein is a method of preparing, separating or purifying the compound of formula (I) or (II).

### Pharmaceutical Composition of the Compound of the Invention and Preparations and Administration

The invention provides a pharmaceutical composition containing a compound of formula (I) or (II) or an independent stereisomer thereof, a racemic mixture or non-racemic mixture of the stereisomer thereof, or a pharmaceutically acceptable salt or solvent thereof. In one embodiment of the invention, the pharmaceutical composition further comprises at least one pharmaceutically acceptable carrier, adjuvant or excipient, and optionally other treating and/or preventing ingredients.

A suitable carrier, adjuvant or excipient is well known for the technical personnel in the field and was described in detail in Ansel H. C. et, al., Ansel's Pharmaceutical Dosage Forms and Drug Delivery Systems (2004) Lippincott, Williams & Wilkins, Philadelphia; Gennaro A. R. et, al., Remington: The Science and Practice of Pharmacy (2000) Lippincott, Williams & Wilkins, Philadelphia; and Rowe R. C., Handbook of Pharmaceutical Excipients (2005) Pharmaceutical Press, Chicago.

“Pharmaceutically acceptable excipient” as used herein means a pharmaceutically acceptable material, composition or vehicle involved in giving form or consistency to the pharmaceutical composition. Each excipient must be compatible with the other ingredients of the pharmaceutical composition when commingled, such that interactions which would substantially reduce the efficacy of the compound of the invention when administered to a patient and would result in pharmaceutically unacceptable compositions are avoided. In addition, each excipient must of course be of sufficiently high purity to render it is pharmaceutically acceptable.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound of the present invention once administered to the patient from one organ, or portion of the body, to another organ, or portion of the body. Certain pharmaceutically acceptable excipients may be chosen for their ability to enhance patient compliance.

Examples of suitable excipients include lactose, glucose, sucrose, sorbitol, mannitol, starch, Arabic gum, calcium phosphate, alginate, xanthate, gelatin, calcium silicate, microcrystalline cellulose, polyvinylpyrrolidone, cellulose, water, syrup and methyl cellulose. Suitable pharmaceutically acceptable excipients further include the following types: diluents, fillers, binders, disintegrants, lubricants (such as talc powder, magnesium stearate and mineral oil), glidants, granulating agents, coating agents, wetting agents, solvents, co-solvents, suspending agents, emulsifiers, sweetners, flavoring agents, flavor masking agents, coloring agents, anticaking agents, humectants, chelating agents, plasticizers, viscosity increasing agents, antioxidants, preservatives (such as methyl hydroxybenzoate and propyl hydroxybenzoate), stabilizers, surfactants and buffering agents. The skilled artisan will appreciate that certain pharmaceutically acceptable excipients may serve more than one function and may serve alternative functions depending on how much of the excipient is present in the formulation and what other ingredients are present in the formulation. The compounds of the invention can be prepared by known methods in the field so that the active components will be released rapidly, continuously or controllably after administration in patients.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

In Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York disclosed various carriers used in formulating pharmaceutically acceptable compositions and known techniques for the preparation thereof, the contents of each of which is incorporated by reference herein. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention.

Suitable pharmaceutically acceptable carriers depend on the pharmaceutical form and are known to those skilled in the art.

As used in the present invention, a “pharmaceutically acceptable carrier” includes any and all solvents and solvent mixtures, coatings, complexing agents, solid carriers, dispersion media, surface active excipients, antibacterial and antifungal drugs, isotonic and absorption delaying agents for pharmaceutically active substances, and mixtures thereof, which are also known in the art.

Some non-limiting examples of pharmaceutically acceptable carriers include those having a component selected from the group consisting of lactose, gelatin, sugar alcohol (e.g., starch, mannitol, corn starch, etc.), vegetable oil, talc, magnesium stearate, colloidal silicon dioxide, carboxymethyl cellulose, microcrystalline cellulose, sodium lauryl sulfate, buffered aqueous solution, copovidone, polysorbate, ethanol, propylene glycol, polyglycol (preferably polyethylene glycol, such as PEG400), Tween®80 (i.e. PEG (20), sorbitol monooleate), DMSO, the mixture of water and cosolvent, for example aqueous solution of alcohol (such as ethanol) and/or polyglycol (such as polyethylene glycol), ester of polyol (such as glycerin and/or polyethylene glycol) and fatty acid, surfactants such as anionic, cationic, nonionic and amphoteric surfactant, complexing agent for example cyclodextrin (e.g. α-cyclodextrin (α-CD) or hydroxypropyl-β-cyclodextrin (HP-β-CD)), bile acid, lipid, such as salts of animal or plant phospholipids, micelle-forming agent, oil such as corn oil and a mixture of two or more of the aforementioned components.

Pharmaceutically acceptable carriers may be solid or liquid carriers for the preparation of pharmaceutical compositions using compounds described in the present invention. Solid formulations include powders, tablets, dispersible granules, capsules, cachets, and suppositories. Powders and tablets may contain about 5% to about 95% active ingredients. Suitable solid carriers are known in the field, such as magnesium carbonate, magnesium stearate, talc powder, sugar or lactose. Tablets, powders, flat capsules and capsules may be used as solid dosage forms suitable for oral administration. Examples of medicinal carriers and methods for preparing various compositions can be obtained as follows: A. Gennaro(ed.), Remington's Pharmaceutical Sciences, 18th ed., 1990, Mack Publishing Company Co., Easton, Pa.

Some non-limiting examples of further suitable pharmaceutically acceptable carriers and suitable additives that can be used in the pharmaceutical composition of the present invention are mentioned below.

In one embodiment, the invention relates to a pharmaceutical composition described herein, which forms a lipid-based drug delivery system (DDS) in an aqueous medium. The pharmaceutical composition includes at least one surfactant in addition to at least one of the compounds having formula (I) or (II) or a salt thereof. Some non-limiting examples of suitable surfactants are as described above. In various embodiments, the lipid-based drug delivery system forms the following structure: (1) liposomes (i.e., dispersed closed bilayer assemblies of lamellar phases in water); (2) nanoparticles of non-lamellar phases (such as cubes, hexagons, sponges); or (3) micelles, emulsion, microemulsion (i.e., simple self-assembling structure of lipids and surfactants).

In some embodiments, lipid-based drug delivery systems that form micelles, emulsions, or microemulsions are preferred. A suitable surfactant or surfactant mixture for forming micelles, emulsions, or microemulsions has a hydrophilic-lipophilic balance (HLB-value) of generally about 8-18, about 10-18, or about 12-16. Lipid-based drug delivery systems form self-emulsifying drug delivery systems (SEDDS) or self-microemulsifying drug delivery systems (SMEDDS). SEDDS and SMEDDS are a mixture of an oil (i.e., a lipid, such as a compound of formula (I) or a salt thereof), at least one surfactant, optionally at least one co-solvent, and optionally at least one co-surfactant. Ideally the mixture is isotropic, when introduced into the water phase under gentle agitation, it spontaneously emulsifies to form an oil-in-water emulsifier. Gentle agitation may be provided, for example, by the mobility of the stomach.

The pharmaceutical compositions of the invention are prepared using techniques and methods known to those skilled in the art. Some of the methods commonly used in the art are described in Remington's Pharmaceutical Sciences (Mack Publishing Company).

Therefore, another aspect of the present invention is related to a method for preparing a pharmaceutical composition, wherein the pharmaceutical composition contains the compound disclosed herein and pharmaceutically acceptable excipient, carrier, adjuvant, vehicle or a combination thereof, and the method comprises mixing various ingredients. The pharmaceutical composition containing the compound disclosed herein can be prepared at for example environment temperature and under barometric pressure.

The compound of the invention will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols, solutions, and dry powders; and (6) topical administration such as creams, ointments, lotions, solutions, pastes, sprays, foams, and gels.

The compounds or pharmaceutical compositions of the invention can be administered in a suitable manner, for example, by the oral, intravenous, subcutaneous, intramuscular or intrathecal route. Preferably it is administered orally, enterally or parenterally. Most preferred is oral administration.

The compounds of the invention can be administered orally, for example, with an inert diluent or with an absorbable food carrier, enclosed in capsules, compressed into tablets, or incorporated directly into dietary foods. For intraoral therapeutic administration, in an exemplary embodiment, the active compound is mixed with excipients and used in the form of absorbable tablet, buccal tablet, tablet, capsule, soft gel capsule, pill, powder, dispersion, lozenge, suspension, syrup, elixir, solution, liquid and the like. Such pharmaceutical compositions and formulations comprise a therapeutically effective amount of the active ingredient, which is usually present at a level of at least 1 wt % of the composition being administered. In various embodiments, the pharmaceutical composition comprises about 5-80 wt % of the active compound.

In various embodiments, tablets, capsules, pills, lozenges and the like contain one or more of the following: excipients such as dicalcium phosphate; lubricants such as magnesium stearate; binding agents such as acacia, tragacanth, corn starch or white gelatin; disintegrating agents such as alginic acid, corn starch, potato starch, etc.; flavoring agents such as wintergreen oil, peppermint; sweetness agents such as saccharin, sucrose or lactose; cherry flavor. When the dosage unit form is a capsule, it may contain, in addition to materials of the above type, a liquid carrier.

Various other materials may exist in the form of a coating or in other form altering the actual form of the dosage unit. For example, tablets, capsules, or pills can be coated with sugar, shellac, or both. The syrup or tincture may contain a compound represented by formula (I) or (II), a dye, a preservative such as methylparaben or propylparaben, a sweetener such as sucrose, and flavorants like cherry or orange spice.

It will also be appreciated that certain of the compounds of present invention can exist in free form for treatment, or where appropriate, as a pharmaceutically acceptable derivative or a prodrug thereof. According to the present invention, a pharmaceutically acceptable derivative includes, but is not limited to, pharmaceutically acceptable prodrugs, salts, esters, salts of such esters, or any other adduct or derivative which upon administration to a patient in need thereof is capable of providing, directly or indirectly, a compound as described herein, or a metabolite or residue thereof.

In one embodiment, the compounds disclosed herein can be prepared to oral administration. In the other embodiment, the compounds disclosed herein can be prepared to inhalation. In the still other embodiment, the compounds disclosed herein can be prepared to nasal administration. In the yet other embodiment, the compounds disclosed herein can be prepared to transdermal administration. In the still yet other embodiments, the compounds disclosed herein can be prepared to topical administration.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

In one embodiment, the compound of formula (I) or (II) is contained in a capsule. Capsule can be hard or soft. Capsules can be made from any suitable film-forming material, including, for example, cellulose derivatives, polyvinyl alcohol, gelatin, pectin, amylopectin or other dextran, modified starches such as starch ethers and oxidized starch, especially hydroxyethylated starch (HES) or hydroxypropylated starch (HPS) alone or a mixture thereof. Cellulose derivatives used to make capsules include, but are not limited to, methyl cellulose, ethyl cellulose, cellulose acetate, hydroxyethyl cellulose, hydroxymethyl cellulose, hydroxypropyl cellulose, hydroxypropylmethyl cellulose, sodium carboxymethyl cellulose, cellulose trimellitate, cellulose acetate phthalate, hydroxypropyl methyl cellulose phthalate, hydroxypropyl methyl cellulose succinate, and a mixture thereof. Preferred cellulose derivatives are methyl cellulose, ethyl cellulose, hydroxyethyl cellulose, hydroxymethyl cellulose, hydroxypropyl cellulose and hydroxypropylmethyl cellulose.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di (lower alkyl) acetal of a lower alkyl aldehyde, e.g., acetaldehyde diethyl acetal; and a water-miscible solvent having one or more hydroxy groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

The pharmaceutical composition of the invention can be prepared to a dosage form adapted for administration to a patient by inhalation, for example as a dry powder, an aerosol, a suspension, or a solution composition. In one embodiment, the pharmaceutical compositions disclosed in the invention is directed to a dosage form adapted for administration to a patient by inhalation as a dry powder. In one embodiment, the pharmaceutical compositions disclosed in the invention is directed to a dosage form adapted for administration to a patient by inhalation as a nebulizer. Dry powder compositions for delivery to the lung by inhalation typically comprise a compound disclosed herein as a finely divided powder together with one or more pharmaceutically-acceptable excipients as finely divided powders. Pharmaceutically-acceptable excipients particularly suited for use in dry powders are known to those skilled in the art and include lactose, starch, mannitol, and mono-, di-, and polysaccharides. The finely divided powder may be prepared by, for example, micronization and milling. Generally, the size-reduced (e.g., micronized) compound can be defined by a D50 value of about 1 to about 10 microns (for example as measured using laser diffraction).

Pharmaceutical compositions adapted for transdermal administration may be presented as discrete patches intended to remain in intimate contact with the epidermis of the patient for a prolonged period of time. For example, the active ingredient may be delivered from the patch by iontophoresis as generally described in Pharmaceutical Research, 3(6), 318(1986).

Pharmaceutical compositions adapted for topical administration may be formulated as ointments, creams, suspensions, lotions, powders, solutions, pastes, gels, sprays, aerosols or oils. Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

The compounds disclosed herein can also be coupled to soluble polymers as targeted medicament carriers. Such polymers may encompass polyvinylpyrrolidone, pyran copolymer, polyhydroxypropylmethacrylamidophenol, polyhydroxyethylaspartamidophenol or polyethylene oxide polylysine substituted by palmitoyl radicals. The compounds may furthermore be coupled to a class of biodegradable polymers which are suitable for achieving controlled release of a medicament, for example polylactic acid, poly-ε-caprolactone, polyhydroxybutyric acid, polyorthoesters, polyacetals, polydihydroxypyrans, polycyanoacrylates and crosslinked or amphipathic block copolymers of hydrogels.

The pharmaceutical compositions provided herein may be administered parenterally by injection, infusion, or implantation, for local or systemic administration. Parenteral administration, as used herein, includes intravenous, intraarterial, intraperitoneal, intrathecal, intraventricular, intraurethral, intrasternal, intracranial, intramuscular, intrasynovial, and subcutaneous administration.

The compounds provided by the invention can also be administered parenterally or intraperitoneally. Dispersions can also be prepared in liquid like polyethylene glycols, glycerol and a mixture thereof, and be prepared in oil. Under ordinary conditions of storage and use, these preparations contain a preservative to prevent the growth of microorganisms.

Pharmaceutical forms suitable for injectable use include sterile aqueous solutions (in the case of water solubility) or dispersions and sterile powders for temporary formulation of sterile injectable solutions or dispersions. In all cases, the form is preferably sterile and fluid, to the extent that injectability is prone to occur. It must be stable under the conditions of manufacture and storage, and must prevent contamination of microorganisms such as bacteria and fungi. The carrier can be a solvent or dispersion medium containing, for example, water, ethanol, polyol (for example, glycerol, propylene glycol, liquid polyethylene glycol, and the like), suitable mixtures thereof, and vegetable oils. For example, proper fluidity can be maintained by maintaining the required particle size (in the case of dispersions), by using a coating such as lecithin, and by using a surfactant. Preventing contamination of microorganisms can be achieved through various antibacterial and antifungal drugs, such as phenol, chlorobutanol, thimerosal, sorbic acid, paraben, etc. In many cases, it will be preferable to include isotonic agents, such as sodium chloride or sugars. Prolonged absorption of injectable compositions can be achieved by using delayed absorption agents such as gelatin and aluminum monostearate in the composition.

A sterile injectable solution is prepared by incorporating a desired amount of a compound of Formula (I) or (II) with a variety of other ingredients listed above in a suitable solvent, and then filtering and sterilizing if necessary. Generally, dispersion is prepared by incorporating various bactericidally active ingredients into a sterile vehicle, which contains a basic dispersion medium and the required other ingredients listed above. In the case of preparing sterile powders for injectable solutions, the preferred methods of preparation are freeze drying and vacuum drying techniques.

The pharmaceutical compositions provided herein may be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration may include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

The pharmaceutical composition provided herein can be administered by rectal in suppository form, in which the drug was mixed with suitable non-irritating excipients such as cocoa oil and glycerol ester synthesized by polyethylene glycol, and the mixture was solid at room temperature and can be released when liquefied or dissolved in the rectal cavity. Because of individual differences, the severity of symptoms between individuals will have great difference, and every drug has its unique therapeutic properties. Therefore, the exact way of administration, dosage form and treatment plan for each individual should be determined by a practicing physician.

The pharmaceutical compositions provided herein may be formulated as immediate or modified release dosage forms, including delayed-, sustained-, pulsed-, controlled-, targeted-, and programmed-release forms. For example, a sustained release dosage form may be considered by incorporating the compound into an ion exchange resin, which may optionally be coated with a diffusion barrier coating to modify the release properties of the resin.

The term “therapeutically effective amount,” as used herein, refers to the total amount of each active component that is sufficient to show a useful treatment effect. For example, the drug amount of administration or balance in the body sufficient to treat, cure, or alleviate symptoms of a disease. The effective amount required for a special treatment depends on a variety of factors, including diseases, the severity of the disease, the activity of the used specific drug, the mode of administration, the clearance rate of the specific drug, the duration of therapy, the combination of drugs, age, weight, gender, diet and patient's health, and so on. The description of other factors that need to be considered for “therapeutically effective amount” in this field can be found in Gilman et, al., eds., Goodman and Gilman's: The Pharmacological Bases of Therapeutics, 8th ed., Pergamon Press, 1990; Remington's Pharmaceutical Sciences, 17th ed., Mack Publishing Company, Easton, Pa., 1990.

For convenient and effective administration, the compound is compounded in an effective amount with a suitable pharmaceutically acceptable carrier and optionally other suitable additives and excipients in the form of dosage units as described above. The dosage of the compound of formula (I) or (II) depends on the route of administration, the age and weight of the patient, the nature and severity of the disease to be treated, and other factors. In various embodiments, the daily dose is typically 2 to 2000 mg/d, such as 50 to 500 mg/d. Within these ranges, in various embodiments, subranges are selected based on lower limit values of 2, 5, 10, 20, 25, 50, 100, 200, 250 or 400 mg/d and upper limit values of 50, 100, 200, 250, 500, 600, 750, 1000, 1500 and 2000 mg/d. The lower and upper limit values can be combined to give a suitable dosage range, which will depend on various factors such as those described above. The daily dose may be administered in a single dosage unit per day or in two or more dosage units per day.

It is particularly advantageous to formulate the pharmaceutical composition of the present invention in dosage unit form to facilitate administration and uniformity of dosage. A dosage unit form as used in the present invention refers to a completely discrete unit suitable as a single dose for a mammal to be treated. Each unit contains a predetermined amount of a compound of Formula (I) or (II), which is designed to produce a desired therapeutic effect together with a required pharmaceutical carrier. The details of the new dosage unit form of the invention are specified by and depend directly on (a) and (b): (a) Unique properties of compounds of Formula (I) or (II) and special therapeutic effects to be obtained; and (b) Limitations inherent in the technology of compounding a compound of formula (I) or (II) for treating a disease in a patient having a disease condition that impairs physical health.

The term “administration” refers to provision of a therapeutically effective amount of medicine to an individual by oral, sublingual, intravenous, subcutaneous, percutaneous, intramuscular, intradermal, intrathecal, epidural, intraocular, intracranial, inhalation, rectal, vagina, etc. The pharmaceutical dosage forms include plaster, lotion, tablet, capsule, pill, dispersible powder, granule, suppository, sublimed preparation, lozenge, injection, aseptic solution or non-aqueous solution, suspension, emulsion, paster, etc. An active component is complexed with a non-toxic pharmaceutically acceptable carrier (such as glucose, lactose, gum arabic, gelatin, mannitol, starch paste, magnesium trisilicate, talcum powder, corn starch, keratin, silica gel, potato starch, urea, dextran, etc.).

The preferred route of administration varies with clinical characteristics. Dose changes must depend on situation of patients receiving treatment. Doctors will determine the appropriate dose according to individual status of patients. The therapeutically effective amount per unit dose depends on body weight, physiological function and the selected vaccination program. An amount of compound per unit dose refers to the weight of the compound per each administration, excluding weight of carriers (the drug formulation contains carriers).

Any suitable route of administration can be used to provide an effective dose of a compound of the invention to a mammal, especially a human. For example, oral administration, rectal administration, parenteral administration, topical administration, ocular administration, nasal administration, pulmonary administration, etc. Dosage forms include tablets, dragees, capsules, creams, ointments, suspensions, dispersions, solutions, aerosols, etc. Preferably, the compound represented of Formula (I) or (II) is administered orally.

The effective dose of active ingredient used may vary with the particular compound used, the route of administration, the symptoms being treated, and the severity of the symptoms being treated. One skilled in the art can easily determine such a dose.

The pharmaceutical compositions provided herein may be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

The pharmaceutical compositions provided herein may be co-formulated with other active ingredients which do not impair the desired therapeutic action, or with substances that supplement the desired action.

In one embodiment, the therapeutic methods disclosed herein comprise administrating to a patient in need of the treatment a safe and effective amount of the compound of the invention or the pharmaceutical composition containing the compound of the invention. Each example disclosed herein comprises the method of treating the diseases comprising administrating to a patient in need of the treatment a safe and effective amount of the compound of the invention or the pharmaceutical composition containing the compound of the invention.

In one embodiment, the compound of the invention or the pharmaceutical composition thereof may be administered by any suitable route of administration, including both systemic administration and topical administration. Systemic administration includes oral administration, parenteral administration, transdermal administration and rectal administration. Typical parenteral administration refers to administration by injection or infusion, including intravenous, intramuscular, and subcutaneous injection or infusion. Topical administration includes application to the skin as well as intraocular, otic, intravaginal, inhaled and intranasal administration. In one embodiment, the compound of the invention or the pharmaceutical composition thereof may be administered orally. In another embodiment, the compound of the invention or the pharmaceutical composition thereof may be administered by inhalation. In still one embodiment, the compound of the invention or the pharmaceutical composition thereof may be administered intranasally.

In one embodiment, the compound of the invention or the pharmaceutical composition thereof may be administered once or according to a dosing regimen wherein a number of doses are administered at varying intervals of time for a given period of time. For example, doses may be administered once, twice, three, or four times per day. In one embodiment, a dose is administered once per day. In a further embodiment, a dose is administered twice per day. Doses may be administered until the desired therapeutic effect is achieved or indefinitely to maintain the desired therapeutic effect. Suitable dosing regimens for the compound of the invention or the pharmaceutical composition thereof depend on the pharmacokinetic properties of that compound, such as absorption, distribution, and half-life, which can be determined by the skilled artisan. In addition, suitable dosing regimens, including the duration of implementation of such regimens, for the compound of the invention or the pharmaceutical composition thereof depend on the disorder being treated, the severity of the disorder being treated, the age and physical condition of the patient being treated, the medical history of the patient to be treated, the nature of concurrent therapy, the desired therapeutic effect, and the like within the knowledge and expertise of the skilled artisan. It will be further understood by such skilled artisans that suitable dosing regimens may require adjustment given an individual patient's response to the dosing regimen or over time as individual patient needs change.

The compounds of the present invention may be administered either simultaneously with, or before or after, one or more other therapeutic agents. The compounds of the present invention may be administered separately, by the same or different route of administration, or together in the same pharmaceutical composition as the other agents. This is chosen by the technical personnel in the field according to the actual conditions of the patient's health, age, weight and so on. If formulated as a fixed dose, such combination products employ the compounds of this invention within the dosage range described herein and the other pharmaceutically active agent or treatment within its dosage range.

Accordingly, in one aspect, this invention includes combinations comprising an amount of at least one compound of the invention, or a pharmaceutically acceptable salt, solvate, ester or prodrug thereof, and an effective amount of one or more additional agents described above.

The compound of Formula (I) or (II) can be used in combination with other drugs for preventing, treating or alleviating diseases or symptoms for which the compound of Formula (I) or (II) is applicable. These other drugs can be administered simultaneously or sequentially with the compound of Formula (I) or (II) by their usual routes and amounts. When the compound of Formula (I) or (II) is used together with one or more other drugs, a pharmaceutical unit dosage form containing such other drugs and the compound of Formula (I) or (II) is preferable.

In various embodiments, the compounds described herein are combined with other drugs to provide a combination therapy for Parkinson's disease or other conditions. The pharmaceutical composition of the present invention includes at least one of the selective adenosine A_{2A} receptor antagonists described in the present invention and an additional therapeutic agent. Examples of the additional therapeutic agent include, but are not limited to:

(1) monoamine oxidase type B inhibitors such as selegiline and rasagiline;

(2) dopamine agonists such as bromocriptine, cabergoline, pergolide, pramipexole, ropinirole and rotigotine;

(3) anticholinergic drugs such as trihexphenidyl, benztropine, orfenadrine, and procyclidine;

(4) glutamine acid antagonists such as amantadine;

(5) levodopa (optionally in combination with carboxylase inhibitor such as carbidopa and benzylhydrazine, or COMT inhibitor such as tocapone and entacapone, or both carboxylase inhibitor and COMT inhibitor).

In all cases, the additional therapeutic agent may be selected from a free base or a neutral compound, or a pharmaceutically acceptable salt. In various embodiments, the selective adenosine A_{2A} receptor antagonists described in the present invention are combined in a single dosage form with one or more additional therapeutic agents. In other embodiments, the selective adenosine A_{2A} receptor antagonist and additional therapeutic agent are combined in a kit form or otherwise provided together for administration.

A method for treating Parkinson's disease or other above-mentioned indications includes administering one or more of selective adenosine A_{2A} receptor antagonists and monoamine oxidase type B inhibitors, dopamine agonists, anticholinergics, glutamate antagonists, and levodopa, wherein non-limiting examples of adjuvant therapeutic agents are those listed above. The selective adenosine A_{2A} receptor antagonist and additional therapeutic agents are administered together in a single dose containing two active ingredients or separately in separate dosage forms, as the case may be. In non-limiting examples, one of the agents is administered in a pill or tablet or other solid dosage form, while the other agents are in a pill or tablet or other solid dosage form, a transdermal patch, or injectable administration.

In one embodiment, the compound of the present invention and the above-mentioned additional therapeutic agent are used in the manufacture of a medicament for preventing, treating or lessening an adenosine A_{2A} receptor-related disease.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of action of the compound in vivo; (b) modify the duration of action of the compound in vivo; (c) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

### Use of the Compounds and Pharmaceutical Compositions

The compounds and pharmaceutical compositions provided by the invention can be used to prepare a medicament for antagonizing adenosine A_{2A} receptor, and also to prepare a medicament for preventing, treating or lessening an adenosine A_{2A} receptor-related disease, especially Parkinson's Disease.

Specifically, the amount of the compound or the compound of the pharmaceutical composition of the present invention can effectively, detectably and selectively antagonize adenosine A_{2A} receptor.

The compounds disclosed herein would be useful for, but are in no way limited to, the prevention or treatment or alleviation of an adenosine A_{2A} receptor-related disease in a patient by administering to the patient a compound or a composition disclosed herein in an effective amount. The adenosine A_{2A} receptor-related diseases further include, but are not limited to: Parkinson's disease, tumour, pain, depression, dementia, stroke, myocardial ischemia, asthma, alcohol withdrawal, dyskinesia syndrome, restless leg syndrome, dystonia, systemic stiffness, neurodegenerative disorders or osteoporosis. Preferably, the compounds of the invention are used for preventing, treating or lessening Parkinson's disease and/or movement disorders.

Besides being useful for human treatment, the compounds and pharmaceutical compositions of the present invention are also useful for veterinary treatment of animals such as companion animals, exotic animals and farm animals. In other embodiments, the animals disclosed herein include horses, dogs, and cats. As used herein, the compounds disclosed herein include the pharmaceutically acceptable derivatives thereof.

### General Synthetic Procedures

The following examples are provided so that the invention might be more fully understood. However, it should be understood that these embodiments merely provide a method of practicing the present invention, and the present invention is not limited to these embodiments.

Generally, the compounds disclosed herein may be prepared by methods described herein, wherein the substituents are as defined for Formula (I) or Formula (II) above, except where further noted. The following non-limiting schemes and examples are presented to further exemplify the invention.

Persons skilled in the art will recognize that the chemical reactions described may be readily adapted to prepare a number of other compounds disclosed herein, and alternative methods for preparing the compounds disclosed herein are deemed to be within the scope disclosed herein. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds disclosed herein.

In the examples described below, unless otherwise indicated all temperatures are set forth in degrees Celsius. Reagents were purchased from commercial suppliers such as Aldrich Chemical Company, Arco Chemical Company and Alfa Chemical Company, and were used without further purification unless otherwise indicated. Common solvents were purchased from commercial suppliers such as Shantou XiLong Chemical Factory, Guangdong Guanghua Reagent Chemical Factory Co. Ltd., Guangzhou Reagent Chemical Factory, Tianjin YuYu Fine Chemical Ltd., Tianjin Fuchen Chemical Reagent Factory, Wuhan XinHuaYuan Technology Development Co. Ltd., Qingdao Tenglong Reagent Chemical Ltd., and Qingdao Ocean Chemical Factory.

Anhydrous THF, dioxane, toluene, and ether were obtained by refluxing the solvent with sodium. Anhydrous CH₂Cl₂ and CHCl₃ were obtained by refluxing the solvent with CaH₂. Ethyl acetate, petroleum ether, n-hexane, N,N-dimethylacetamide and N,N-dimethylformamide were treated with anhydrous sodium sulfate prior to use.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was conducted using a silica gel column. Silica gel (300-400 mesh) was purchased from Qingdao Ocean Chemical Factory.

¹H NMR spectra were recorded by Bruker 400 MHz or 600 MHz NMR spectrometer. ¹H NMR spectra were obtained by using CDCl₃, DMSO-d₆, CD₃OD or acetone-d₆ solutions (in ppm), with TMS (0 ppm) or chloroform (7.26 ppm) as the reference standard. When peak multiplicities are reported, the following abbreviations are used: s (singlet), d (doublet), t (triplet), q (quartet), m (multiplet), br (broadened), brs (broadened singlet), dd (doublet of doublets), ddd (doublet of doublet of triplets), ddt (doublet of doublet of triplets), dt (doublet of triplets), dq (doublet of quartets), td (triplet of doublets), tt (triplet of triplets), qd (quartet of doublets). Coupling constants J, when given, were reported in Hertz (Hz).

Low resolution mass spectrum (MS) data measurement condition: Agilent 6120 Quadrupole HPLC-MS (column type: Zorbax SB-C18, 2.1×30 mm, 3.5 micron, 6 min, flow rate 0.6 mL/min). Mobile phase: in the proportion of 5%-95% (CH₃CN containing 0.1% of formic acid) in (H₂O containing 0.1% of formic acid), using electrospray ionization (ESI), UV detection, at 210 nm/254 nm.

Pure compound was detected by Agilent 1260 pre-HPLC or Calesep pump 250 pre-HPLC (NOVASEP 50/80 mm DAC) with UV detection at 210/254 nm.

The following abbreviations are used throughout the specification:

| | | | |
|---|---|---|---|
| CDCl₃ | deuterochloroform | mg | milligram |
| DMSO | dimethyl sulfoxide | g | gram |
| DMSO-d₆ | deuterated | kg | kilogram |
| | dimethyl sulfoxide | | |
| CH₃OH, | methanol | mL, ml | milliliter |
| MeOH | | | |
| H₂O | water | μL, μl | microliter |
| HCOONH₄ | ammonium formate | nL, nl | nanoliter |
| FA | formic acid | s | second |
| nM, | nanomole per liter | min | minute |
| nmol/L | | | |
| μM, | micromole per liter | h | hour |
| μmol/L | | | dipotassium |
| mM, | millimole per liter | EDTA-K₂ | ethylenediaminetetraacetate |
| mmol/L | | | |
| M, mol/L | moles per liter | cAMP | cyclic adenosine |
| | | | monophosphate |
| mmol | millimole | PEG400 | polyethylene glycol 400 |
| ng | nanogram | DMA | N,N-dimethylaniline |
| μg | microgramme | Saline | Saline |
| PBS | phosphate buffer | | |
| | saline | | |
| HBSS | Hank′s Balanced | | |
| | Salt Solution | | |

| | |
|---|---|
| HATU | 2-(7-benzotriazole oxide)-N,N,N′,N′-tetramethylurea |
| | hexafluorophosphate |
| NECA | (2S, 3S, 4R, 5S)-5-(6-amino-9H-purin-9-yl)-N- |
| | ethyl-3,4-dihydroxytetrahydrofuran-2-carboxamide, |
| | C₁₂H₁₆N₆O₄ adenosine receptor agonist, |
| | 5-(N-ethylcarboxamide)-adenosine |

The following intermediate preparation schemes and synthetic schemes describe the steps for preparing the compounds disclosed herein, unless otherwise specified, wherein R⁷ is as defined herein.

### Intermediate Preparation Scheme 1

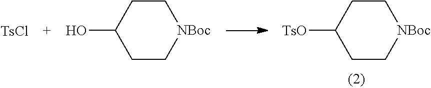

The compound (2) can be prepared by the following process: p-toluenesulfonyl chloride can react with tert-butyl 4-hydroxypiperidine-1-carboxylate to give a compound (2).

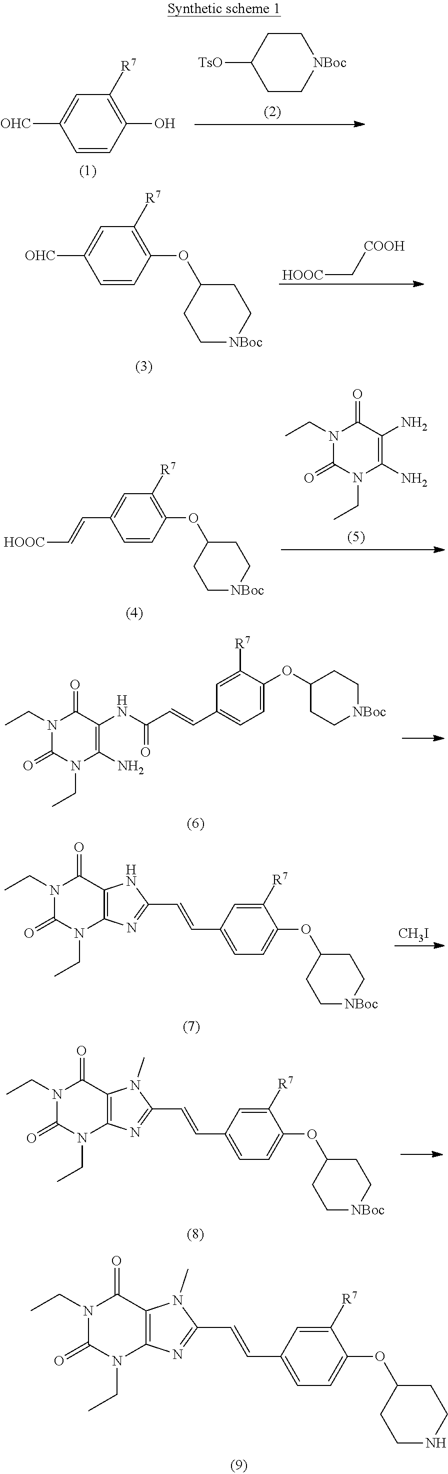

Compound (9) can be prepared through the following process: compound (1) can react with compound (2) to give a compound 3); compound (3) can react with malonic acid to give a compound (4); compound (4) can react with compound (5) to give a compound (6); and then compound (6) can undergo ring-closing to give a compound (7). Compound (7) can react with methyl iodide to give a compound (8). Compound (8) can remove the Boc protecting group to give the target product of compound (9).

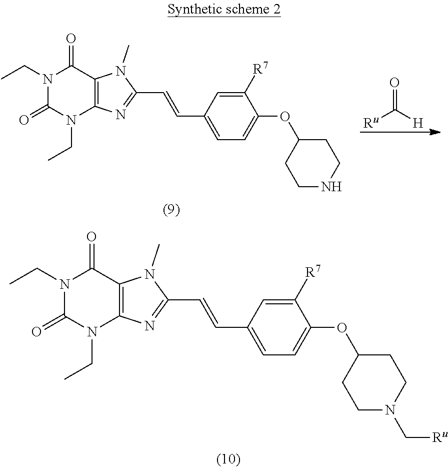

Wherein, R^{u} is H, C₁-C₆ alkyl, C₁-C₆ haloalkyl or C₃-C₈ cycloalkyl.

Compound (10) can be prepared through the following process: the compound (9) can undergo a Borch reductive amination reaction with the corresponding aldehyde

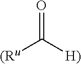

in the presence of a reducing agent (for example, sodium cyanoborohydride) to give the target product of compound (10).

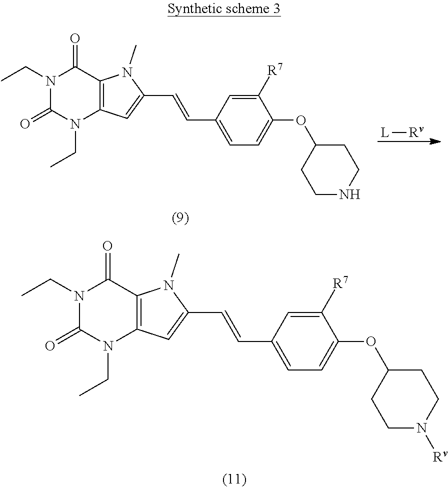

Wherein, L is a leaving group, such as

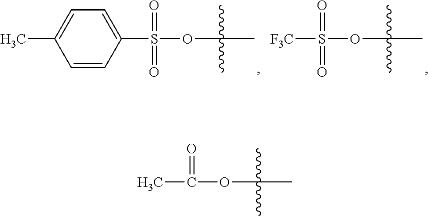

or halogen (F, Cl, Br, I), etc.; R^{v} is C₁-C₆ alkyl, C₁-C₆ haloalkyl, —C(═O)—(C₁-C₆ alkyl), —C(═O)—(C₃-C₈ cycloalkyl) or —S(═O)₂—(C₁-C₆ alkyl).

Compound (11) can be prepared through the following process: the compound (9) can undergo a nucleophilic substitution reaction with the compound L-R^{v} to give the target product of compound (11).

The following examples are provided to further illustrate the compounds, pharmaceutical compositions and their applications thereof.

### EXAMPLE

### Example 1 Synthesis of (E)-1,3-diethyl-7-methyl-8-(4-(piperidin-4-yloxy)styryl)-1H-purine-2,6(3H,7H)-dione

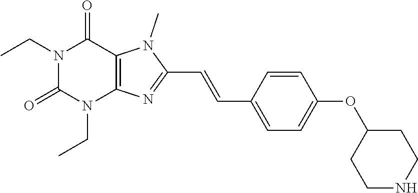 [SMILES: CCn1c(=O)c2c(nc(/C=C/c3ccc(OC4CCNCC4)cc3)n2C)n(CC)c1=O]

### Step 1) Synthesis of tert-butyl 4-(p-toluenesulfonyloxy)piperidine-1-carboxylate

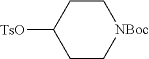 [SMILES: Cc1ccc(S(=O)(=O)OC2CCN(C(=O)OC(C)(C)C)CC2)cc1]

To a 100 mL single-necked flask were added p-toluenesulfonyl chloride (7.1 g, 37.0 mmol) and tert-butyl 4-hydroxypiperidine-1-carboxylate (5.0 g, 24.8 mmol) at 25° C. Dichloromethane (40 mL) was added, then triethylamine (10.4 mL, 74.2 mmol) was added dropwise, and the reaction was continued for 18 h. After that, saturated sodium bicarbonate solution (30 mL) was added. The reaction solution was partitioned. The organic phase was collected, spin-dried under reduced pressure and purified by column chromatography (petroleum ether/ethyl acetate (v/v)=10/1˜5/1) to give the title compound as a white solid (5.6 g, 63%).

MS (ESI, pos. ion) m/z: 300.0 [M+H−56]⁺;

¹H NMR (400 MHz, CDCl₃) δ (ppm) 7.82 (d, J=8.2 Hz, 2H), 7.36 (d, J=8.1 Hz, 2H), 4.70 (tt, J=7.3, 3.7 Hz, 1H), 3.65-3.57 (m, 2H), 3.31-3.23 (m, 2H), 2.47 (s, 3H), 1.84-1.75 (m, 2H), 1.70 (dt, J=13.2, 7.5 Hz, 2H), 1.45 (s, 9H).

### Step 2) Synthesis of tert-butyl 4-(4-formylphenoxy)piperidine-1-carboxylate

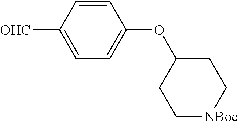 [SMILES: CC(C)(C)OC(=O)N1CCC(Oc2ccc(C=O)cc2)CC1]

To a 100 mL single-necked round bottom flask were added p-hydroxybenzaldehyde (2.5 g, 20.5 mmol), tert-butyl 4-(p-toluenesulfonyloxy) piperidine-1-carboxylate (5.6 g, 15.8 mmol) and N,N-dimethylformamide (30 mL). Cesium Carbonate (10 g, 30.7 mmol) was added and the mixture was stirred at 85° C. in an oil bath for 3 h. The reaction solution was quenched with water (300 mL). Then ethyl acetate (50 mL) was added, and the resulting mixture was separated. The organic phase was collected, spin-dried under reduced pressure. The residue was purified by column chromatography (petroleum ether/ethyl acetate (v/v)=4/1) to give the title compound as colourless oil (3.5 g, 73%).

MS (ESI, pos. ion) m/z: 250.1 [M+H−56]⁺;

¹H NMR (400 MHz, CDCl₃) δ (ppm) 9.88 (s, 1H), 7.83 (d, J=8.7 Hz, 2H), 7.01 (d, J=8.7 Hz, 2H), 4.61 (tt, J=6.9, 3.4 Hz, 1H), 3.74-3.66 (m, 2H), 3.42-3.34 (m, 2H), 2.00-1.91 (m, 2H), 1.79 (ddd, J=13.5, 7.3, 3.9 Hz, 2H), 1.47 (s, 9H).

### Step 3) synthesis of (E)-3-(4-((1-(tert-butoxycarbonyl)piperidin-4-yl)oxy)phenyl)acrylic acid

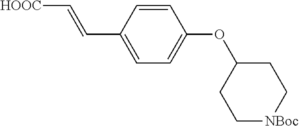 [SMILES: CC(C)(C)OC(=O)N1CCC(Oc2ccc(/C=C/C(=O)O)cc2)CC1]

To a 100 mL single-necked round bottom flask were added tert-butyl 4-(4-formylphenoxy) piperidine-1-carboxylate (3.5 g, 11.5 mmol), malonic acid (1.8 g, 17.3 mmol), piperidine (1.1 mL, 12 mmol) and pyridine (10 mL). The mixture was stirred at 100° C. in an oil bath for 4.5 h. After the reaction, the mixture was cooled to room temperature and spin-dried under reduced pressure. The residue was purified by column chromatography (PE/EtOAc (v/v)=2/1) to give the title compound as a white solid (3.9 g, 97.7%).

MS (ESI, pos. ion) m/z: 292.3 [M+H−56]⁺;

¹H NMR (400 MHz, CDCl₃) δ (ppm) 7.74 (d, J=15.9 Hz, 1H), 7.51 (d, J=8.6 Hz, 2H), 6.93 (d, J=8.7 Hz, 2H), 6.34 (d, J=15.9 Hz, 1H), 4.58-4.52 (m, 1H), 3.75-3.67 (m, 2H), 3.42-3.34 (m, 2H), 2.00-1.90 (m, 2H), 1.78 (dt, J=12.9, 7.1 Hz, 2H), 1.49 (s, 9H).

### Step 4) (E)-tert-butyl 4-(4-(3-((6-amino-1,3-diethyl-2,4-dioxo-1,2,3,4-tetrahydropyrimidine-5-yl) amino)-3-oxoprop-1-en-1-yl)phenoxy)piperidine-1-carboxylate

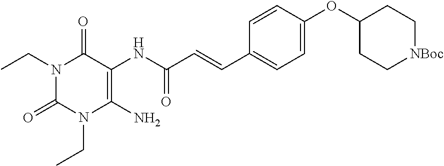 [SMILES: CCn1c(N)c(NC(=O)/C=C/c2ccc(OC3CCN(C(=O)OC(C)(C)C)CC3)cc2)c(=O)n(CC)c1=O]

To a 100 mL single-necked round bottom flask were added (E)-3-(4-((1-(tert-butoxycarbonyl)piperidin-4-yl)oxy) phenyl)acrylic acid (3.9 g, 11.2 mmol) and dichloromethane (40 mL) at 0° C. N,N-diisopropylethylamine (7.6 mL, 46 mmol) and HATU (5.5 g, 14 mmol) were added. The mixture was continued stirring for 0.5 h. 5,6-Diamino-1,3-diethylpyrimidine-2,4 (1H,3H)-dione hydrochloride (3.5 g, 15 mmol) was then added. The reaction solution was transferred to 25° C. and reacted for 4 h. Water (30 mL) and dichloromethane (30 mL) were added. The reaction solution was separated. The organic phase was collected, spin-dried under reduced pressure and the residue was purified by column chromatography (DCM/MeOH (v/v)=20/1) to give the title compound as as a yellow solid (5.6 g, 94.9%).

MS (ESI, pos. ion) m/z: 528.1 [M+H]⁺;

¹H NMR (400 MHz, CDCl₃) δ (ppm) 7.85 (s, 1H), 7.56 (d, J=15.5 Hz, 1H), 7.42 (d, J=8.5 Hz, 2H), 6.87 (d, J=8.5 Hz, 2H), 6.58 (d, J=15.5 Hz, 1H), 5.79 (s, 2H), 4.55-4.46 (m, 1H), 4.02-3.92 (m, 4H), 3.74-3.66 (m, 2H), 3.40-3.31 (m, 2H), 1.92 (s, 2H), 1.80-1.70 (m, 2H), 1.48 (s, 9H), 1.31 (t, J=7.1 Hz, 3H), 1.20 (t, J=7.0 Hz, 3H).

### Step 5) Synthesis of (E)-tert-butyl 4-(4-(2-(1,3-diethyl-2,6-dioxo-2,3,6,7-tetrahydro-1H-purin-8-yl)vinyl)phenoxy)piperidine-1-carboxylate

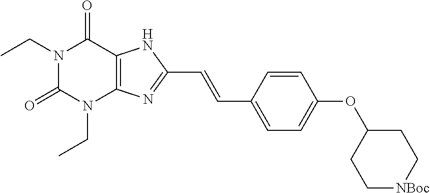 [SMILES: CCn1c(=O)c2[nH]c(/C=C/c3ccc(OC4CCN(C(=O)OC(C)(C)C)CC4)cc3)nc2n(CC)c1=O]

To a 100 mL single-necked round bottom flask were added (E)-tert-butyl 4-(4-(3-((6-amino-1,3-diethyl-2,4-dioxo-1,2,3,4-tetrahydropyrimidine-5-yl)amino)-3-oxoprop-1-en-1-yl)phenoxy)piperidine-1-carboxylate (5.6 g, 10.6 mmol) and methanol (40 mL). Then water (10 mL) and sodium hydroxide (1.3 g, 32.5 mmol) were added. The mixture was stirred at 70° C. in an oil bath for 14 h. The reaction solution was stopped, and spin-dried under reduced pressure. Water (20 mL) and dichloromethane (50 mL) were added. The resulting mixture was separated. The organic phase was collected, spin-dried under reduced pressure. Acetone (15 mL) was added. The mixture was stirred in slurry for 15 minutes, filtered and the filter cake was dried to give the title compound as a yellow solid (3.5 g, 64.8%).

MS (ESI, pos. ion) m/z: 510.2 [M+H]⁺;

¹H NMR (400 MHz, DMSO-d₆) δ (ppm) 7.52 (d, J=8.6 Hz, 2H), 7.45 (d, J=16.3 Hz, 1H), 6.99 (d, J=8.6 Hz, 2H), 6.89 (d, J=16.3 Hz, 1H), 4.64-4.57 (m, 1H), 4.05 (dd, J=13.8, 6.8 Hz, 2H), 3.92 (dd, J=13.8, 6.8 Hz, 2H), 3.70-3.64 (m, 4H), 1.90 (d, J=5.1 Hz, 2H), 1.53 (dt, J=13.2, 6.2 Hz, 2H), 1.41 (s, 9H), 1.24 (t, J=7.0 Hz, 3H), 1.12 (t, J=6.9 Hz, 3H).

### Step 6) Synthesis of (E)-tert-butyl 4-(4-(2-(1,3-diethyl-7-methyl-2,6-dioxo-2,3,6,7-tetrahydro-1H-purin-8-yl)vinyl)phenoxy)piperidine-1-carboxylate

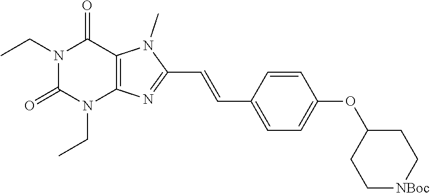 [SMILES: CCn1c(=O)c2c(nc(/C=C/c3ccc(OC4CCN(C(=O)OC(C)(C)C)CC4)cc3)n2C)n(CC)c1=O]

To a 100 mL single-necked round bottom flask were added (E)-tert-butyl 4-(4-(2-(1,3-diethyl-2,6-dioxo-2,3,6,7-tetrahydro-1H-purin-8-yl)vinyl) phenoxy)piperidine-1-carboxylate (3.5 g, 6.9 mmol) and N,N-dimethylformamide (40 mL) at 0° C. Cesium carbonate (5.6 g, 17 mmol) was added. The resulting solution was stirred for 10 min, methyl iodide (0.86 mL, 14 mmol) was added, and then the mixture was transferred to 25° C. and stirred for 3 h. The reaction was stopped, and water (100 mL) was added. The resulting solution was extracted with dichloromethane (50 mL) and separated. The organic phase was spin-dried under reduced pressure. The residue was purified by column chromatography (petroleum ether/ethyl acetate (v/v)=5/1) to give the title compound as a light yellow solid (3.5 g, 97%).

MS (ESI, pos. ion) m/z: 524.1 [M+H]⁺;

¹H NMR (400 MHz, CDCl₃) δ (ppm) 7.75 (d, J=15.7 Hz, 1H), 7.54 (d, J=8.6 Hz, 2H), 6.95 (d, J=8.7 Hz, 2H), 6.79 (d, J=15.7 Hz, 1H), 4.58-4.52 (m, 1H), 4.23 (q, J=7.0 Hz, 2H), 4.12 (dd, J=14.0, 7.1 Hz, 2H), 4.06 (s, 3H), 3.76-3.68 (m, 2H), 3.42-3.34 (m, 2H), 2.00-1.91 (m, 2H), 1.79 (dt, J=13.1, 7.1 Hz, 2H), 1.49 (s, 9H), 1.40 (t, J=7.0 Hz, 3H), 1.28 (t, J=7.0 Hz, 3H).

### Step 7) Synthesis of (E)-1,3-diethyl-7-methyl-8-(4-(piperidin-4-yloxy)styryl)-1H-purine-2,6(3H,7H)-dione

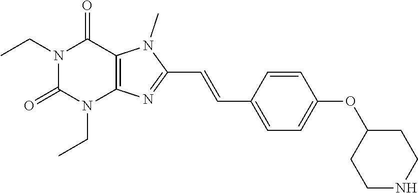 [SMILES: CCn1c(=O)c2c(nc(/C=C/c3ccc(OC4CCNCC4)cc3)n2C)n(CC)c1=O]

To a 100 mL single-necked round bottom flask were added (E)-tert-butyl 4-(4-(2-(1,3-diethyl-7-methyl-2,6-dioxo-2,3,6,7-tetrahydro-1H-purin-8-yl)vinyl)phenoxy)piperidine-1-carboxylate (3.5 g, 6.7 mmol) and dichloromethane (25 mL) at 25° C. Methanesulfonic acid (1.3 mL, 20 mmol) was added. The resulting solution was stirred for 2 hours. Saturated sodium bicarbonate solution was added to adjust the solution to pH 8. Then the solution was separated, and the organic phase was collected, dried with anhydrous sodium sulfate (2.0 g), filtered, and concentrated to give the title compound as a light yellow solid (2.8 g, 99%).

MS (ESI, pos. ion) m/z: 424.2 [M+H]⁺;

¹H NMR (400 MHz, CDCl₃) δ (ppm) 7.75 (d, J=15.7 Hz, 1H), 7.53 (d, J=8.7 Hz, 2H), 6.95 (d, J=8.7 Hz, 2H), 6.78 (d, J=15.7 Hz, 1H), 4.50-4.43 (m, 1H), 4.23 (q, J=7.1 Hz, 2H), 4.10 (q, J=7.0 Hz, 2H), 4.06 (s, 3H), 3.22-3.15 (m, 2H), 2.83-2.79 (m, 2H), 2.07 (d, J=6.5 Hz, 2H), 1.78-1.68 (m, 2H), 1.40 (t, J=7.1 Hz, 3H), 1.28 (t, J=7.0 Hz, 3H).

### Example 2 Synthesis of (E)-1,3-diethyl-7-methyl-8-(4-((1-methylpiperidin-4-yl)oxy)styryl)-1H-purine-2,6(3H,7H)-dione

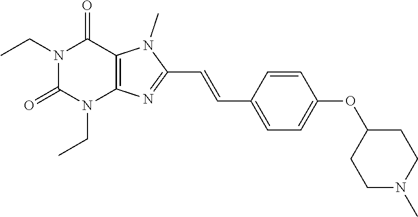 [SMILES: CCn1c(=O)c2c(nc(/C=C/c3ccc(OC4CCN(C)CC4)cc3)n2C)n(CC)c1=O]

To a 100 mL single-necked round bottom flask were added (E)-1,3-diethyl-7-methyl-8-(4-(piperidin-4-yloxy)styryl)-1H-purine-2,6(3H,7H)-dione (0.5 g, 1.18 mmol) and methanol (10 mL) at 0° C. Formaldehyde aqueous solution (0.88 mL, 12 mmol, 37 mass %) and acetic acid (142 mg, 2.36 mmol) were added. The resulting solution was stirred for 15 min. Then sodium cyanoborohydride (227 mg, 3.54 mmol) was added, and transferred to 25° C. and stirred for 12 h. The reaction was stopped, and water (30 mL) was added. The resulting solution was extracted with dichloromethane (50 mL) and separated. The organic phase was spin-dried under reduced pressure. The residue was purified by column chromatography (dichloromethane/methanol (v/v)=15/1) to give the title compound as an off-white solid (0.49 g, 94.8%).

MS (ESI, pos. ion) m/z: 438.1 [M+H]⁺;

¹H NMR (400 MHz, CDCl₃) δ (ppm) 7.75 (d, J=15.7 Hz, 1H), 7.54 (d, J=8.6 Hz, 2H), 6.94 (d, J=8.6 Hz, 2H), 6.79 (d, J=15.7 Hz, 1H), 4.46-4.39 (m, 1H), 4.23 (q, J=7.0 Hz, 2H), 4.11 (q, J=7.1 Hz, 2H), 4.06 (s, 3H), 3.03 (q, J=9.6 Hz, 2H), 2.97-2.90 (m, 2H), 2.70-2.62 (m, 2H), 2.08-2.00 (m, 2H), 1.89 (ddd, J=12.7, 7.5, 3.6 Hz, 2H), 1.40 (t, J=7.0 Hz, 3H), 1.28 (t, J=7.0 Hz, 3H).

### Example 3 Synthesis of (E)-1,3-diethyl-7-methyl-8-(4-((1-ethylpiperidin-4-yl)oxy)styryl)-1H-purine-2,6(3H,7H)-dione

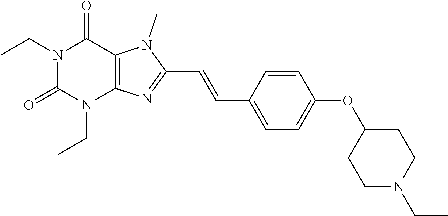 [SMILES: CCN1CCC(Oc2ccc(/C=C/c3nc4c(c(=O)n(CC)c(=O)n4CC)n3C)cc2)CC1]

To a 50 mL single-necked round bottom flask were added (E)-1,3-diethyl-7-methyl-8-(4-(piperidin-4-yloxy)styryl)-1H-purine-2,6(3H,7H)-dione (500 mg, 1.18 mmol) and acetonitrile (5 mL) at 25° C. N,N-Diisopropylethylamine (0.56 mL, 3.39 mmol) was added. Then iodoethane (0.191 mL, 2.38 mmol) was added dropwise. The resulting solution was stirred for 3 h. The reaction was stopped, and spin-dried under reduced pressure. The residue was purified by column chromatography (dichloromethane/methanol (v/v)=30/1) to give the title compound as a light yellow solid (0.455 g, 85.3%).

MS (ESI, pos. ion) m/z: 452.2 [M+H]⁺;

¹H NMR (400 MHz, CDCl₃) δ (ppm) 7.75 (d, J=15.7 Hz, 1H), 7.57 (d, J=8.6 Hz, 2H), 6.95 (d, J=8.6 Hz, 2H), 6.81 (d, J=15.7 Hz, 1H), 4.81 (brs, 1H), 4.22 (q, J=6.9 Hz, 2H), 4.15-4.03 (m, 5H), 3.47 (brs, 2H), 3.30-3.08 (m, 4H), 2.77 (brs, 2H), 2.34-2.18 (m, 2H), 1.57 (t, J=7.3 Hz, 3H), 1.48 (t, J=7.3 Hz, 3H), 1.39 (t, J=7.0 Hz, 3H).

### Example 4 Synthesis of (E)-8-(4-((1-(2,2-difluoroethyl)piperidin-4-yl)oxy)styryl)-1,3-diethyl-7-methyl-1H-purine-2,6(3H,7H)-dione

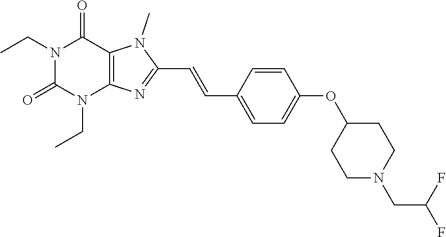 [SMILES: CCn1c(=O)c2c(nc(/C=C/c3ccc(OC4CCN(CC(F)F)CC4)cc3)n2C)n(CC)c1=O]

To a 100 mL single-necked round bottom flask were added (E)-1,3-diethyl-7-methyl-8-(4-(piperidin-4-yloxy)styryl)-1H-purine-2,6(3H,7H)-dione (500 mg, 1.18 mmol), potassium carbonate (495 mg, 3.55 mmol), sodium iodide (177 mg, 1.18 mmol) and acetonitrile (20 mL). 2,2-Difluoroethyl p-toluenesulfonate (837 mg, 3.54 mmol) was added. The resulting solution was reacted for 20 hours at 90° C. in an oil bath. The reaction was stopped, and saturated sodium bicarbonate solution (20 mL) was added. The mixture was extracted with dichloromethane (30 mL) and separated. The organic phase was spin-dried under reduced pressure. The residue was purified by column chromatography (petroleum ether/ethyl acetate (v/v)=1/1) to give the title compound as an off-white solid (0.35 g, 60.8%).

MS (ESI, pos. ion) m/z: 488.1 [M+H]⁺;

¹H NMR (400 MHz, CDCl₃) δ (ppm) 7.73 (d, J=15.7 Hz, 1H), 7.51 (d, J=8.6 Hz, 2H), 6.92 (d, J=8.6 Hz, 2H), 6.76 (d, J=15.7 Hz, 1H), 5.89 (tt, J=56.0, 4.2 Hz, 1H), 4.43-4.36 (m, 1H), 4.21 (q, J=7.0 Hz, 2H), 4.08 (q, J=7.0 Hz, 2H), 4.04 (s, 3H), 2.88-2.72 (m, 4H), 2.56-2.48 (m, 2H), 2.05-1.97 (m, 2H), 1.91-1.82 (m, 2H), 1.38 (t, J=7.0 Hz, 3H), 1.26 (t, J=7.0 Hz, 3H).

### Example 5 Synthesis of (E)-1,3-diethyl-7-methyl-8-(4-((1-(2,2,2-trifluoroethyl)piperidin-4-yl)oxy)styryl)-1H-purine-2,6(3H,7H)-dione

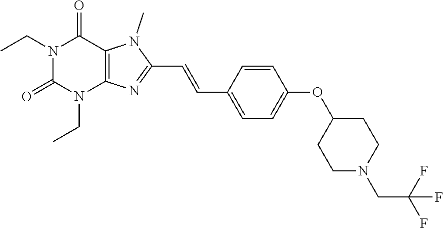 [SMILES: CCn1c(=O)c2c(nc(/C=C/c3ccc(OC4CCN(CC(F)(F)F)CC4)cc3)n2C)n(CC)c1=O]

To a 100 mL single-necked round bottom flask were added (E)-1,3-diethyl-7-methyl-8-(4-(piperidin-4-yloxy)styryl)-1H-purine-2,6(3H,7H)-dione (450 mg, 1.06 mmol), N,N-diisopropylethylamine (1.1 mL, 6.6 mmol) and dichloromethane (8 mL) at 25° C. 2,2,2-Trifluoroethyl trifluoromethanesulfonate (0.38 mL, 2.6 mmol) was added. The resulting solution was reacted for 12 hours. The reaction was stopped, and saturated sodium bicarbonate solution (20 mL) and dichloromethane (30 mL) was added for extraction and separation. The organic phase was spin-dried under reduced pressure. The residue was purified by column chromatography (petroleum ether/ethyl acetate (v/v)=1/1) to give the title compound as an off-white solid (0.33 g, 61.4%).

MS (ESI, pos. ion) m/z: 506.1 [M+H]⁺;

¹H NMR (400 MHz, CDCl₃) δ (ppm) 7.75 (d, J=15.7 Hz, 1H), 7.54 (d, J=8.6 Hz, 2H), 6.94 (d, J=8.6 Hz, 2H), 6.79 (d, J=15.7 Hz, 1H), 4.46-4.39 (m, 1H), 4.23 (q, J=7.0 Hz, 2H), 4.11 (q, J=7.1 Hz, 2H), 4.06 (s, 3H), 3.03 (q, J=9.6 Hz, 2H), 2.97-2.90 (m, 2H), 2.70-2.62 (m, 2H), 2.08-2.00 (m, 2H), 1.89 (ddd, J=12.7, 7.5, 3.6 Hz, 2H), 1.40 (t, J=7.0 Hz, 3H), 1.28 (t, J=7.0 Hz, 3H).

### Example 6 Synthesis of (E)-8-(4-((1-acetylpiperidin-4-yl)oxy)styryl)-1,3-diethyl-7-methyl-1H-purine-2,6 (3H,7H)-dione

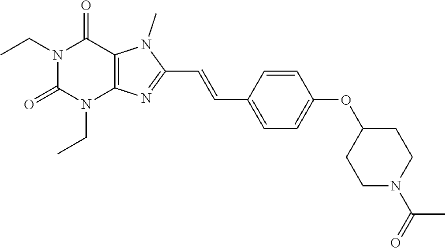 [SMILES: CCn1c(=O)c2c(nc(/C=C/c3ccc(OC4CCN(C(C)=O)CC4)cc3)n2C)n(CC)c1=O]

To a 50 mL single-necked round bottom flask were added (E)-1,3-diethyl-7-methyl-8-(4-(piperidin-4-yloxy)styryl)-1H-purine-2,6(3H,7H)-dione (0.5 g, 1.18 mmol), triethylamine (0.5 mL, 3.6 mmol) and dichloromethane (8 mL) at 0° C. Acetic anhydride (0.22 mL, 2.3 mmol) was added. The resulting solution was reacted for 5 hours. The reaction was stopped, and saturated sodium bicarbonate solution (20 mL) and dichloromethane (20 mL) was added for extraction and separation. The organic phase was spin-dried under reduced pressure. The residue was purified by column chromatography (petroleum ether/ethyl acetate (v/v)=1/1) to give the title compound as an off-white solid (0.53 g, 96.4%).

MS (ESI, pos. ion) m/z: 466.4 [M+H]⁺;

¹H NMR (600 MHz, CDCl₃) δ (ppm) 7.76 (d, J=15.7 Hz, 1H), 7.55 (d, J=8.7 Hz, 2H), 6.96 (d, J=8.7 Hz, 2H), 6.80 (d, J=15.7 Hz, 1H), 4.65-4.61 (m, 1H), 4.23 (q, J=7.1 Hz, 2H), 4.11 (q, J=7.0 Hz, 2H), 4.06 (s, 3H), 3.80-3.76 (m, 1H), 3.75-3.68 (m, 2H), 3.48-3.44 (m, 1H), 2.15 (s, 3H), 2.01-1.93 (m, 2H), 1.92-1.82 (m, 2H), 1.40 (t, J=7.1 Hz, 3H), 1.28 (t, J=7.1 Hz, 3H).

### Example 7 Synthesis of (E)-8-(4-((1-(cyclopropenyl)piperidin-4-yl)oxy)styryl)-1,3-diethyl-7-methyl-1H-purine-2,6(3H, 7H)-dione

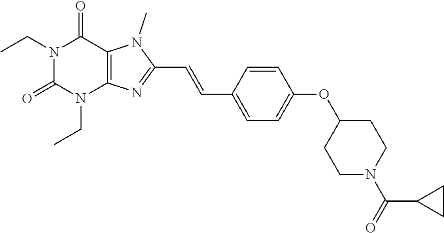 [SMILES: CCn1c(=O)c2c(nc(/C=C/c3ccc(OC4CCN(C(=O)C5CC5)CC4)cc3)n2C)n(CC)c1=O]

To a 50 mL single-necked round bottom flask were added (E)-1,3-diethyl-7-methyl-8-(4-(piperidin-4-yloxy)styryl)-1H-purine-2,6(3H,7H)— dione (0.52 g, 1.23 mmol), triethylamine (0.7 mL, 5.1 mmol) and dichloromethane (8 mL) at 0° C. Cyclopropylformyl chloride (0.22 mL, 2.4 mmol) was added. The resulting solution was transferred to 25° C. and continued to react for 1 h. The reaction was stopped, and saturated sodium bicarbonate solution (20 mL) and dichloromethane (20 mL) was added for extraction and separation. The organic phase was spin-dried under reduced pressure. The residue was purified by column chromatography (petroleum ether/ethyl acetate (v/v)=1/1) to give the title compound as an off-white solid (0.6 g, 99.4%).

MS (ESI, pos. ion) m/z: 492.2 [M+H]⁺;

¹H NMR (400 MHz, CDCl₃) δ (ppm) 7.74 (d, J=15.7 Hz, 1H), 7.53 (d, J=8.6 Hz, 2H), 6.94 (d, J=8.6 Hz, 2H), 6.78 (d, J=15.7 Hz, 1H), 4.69-4.62 (m, 1H), 4.21 (q, J=7.0 Hz, 2H), 4.08 (q, J=7.0 Hz, 2H), 4.06 (s, 3H), 3.97-3.75 (m, 2H), 3.72-3.61 (m, 2H), 1.99-1.84 (m, 4H), 1.82-1.76 (m, 1H), 1.38 (t, J=7.0 Hz, 3H), 1.26 (t, J=7.0 Hz, 3H), 1.00 (dd, J=6.9, 4.0 Hz, 2H), 0.77 (dd, J=7.8, 3.0 Hz, 2H).

### Example 8 Synthesis of (E)-1,3-diethyl-7-methyl-8-(4-((1-(methylsulfonyl)piperidin-4-yl)oxy)styryl)-1H-purine-2,6(3H, 7H)-dione

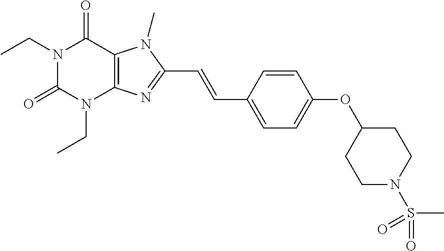 [SMILES: CCn1c(=O)c2c(nc(/C=C/c3ccc(OC4CCN(S(C)(=O)=O)CC4)cc3)n2C)n(CC)c1=O]

To a 50 mL single-necked round bottom flask were added (E)-1,3-diethyl-7-methyl-8-(4-(piperidin-4-yloxy)styryl)-1H-purine-2,6(3H,7H)-dione (0.5 g, 1.18 mmol), triethylamine (0.5 mL, 3.6 mmol) and dichloromethane (10 mL) at 0° C. Methanesulfonyl chloride (0.2 g, 1.75 mmol) was added. The resulting solution was transferred to 25° C. and reacted for 6 hours. The reaction was stopped, and saturated sodium bicarbonate solution (20 mL) and dichloromethane (20 mL) was added for extraction and separation. The organic phase was spin-dried under reduced pressure. The residue was purified by column chromatography (petroleum ether/ethyl acetate (v/v)=1/1) to give the title compound as a light yellow solid (0.546 g, 92.4%).

MS (ESI, pos. ion) m/z: 502.2 [M+H]⁺;

¹H NMR (400 MHz, CDCl₃) δ (ppm) 7.72 (d, J=15.7 Hz, 1H), 7.53 (d, J=8.7 Hz, 2H), 6.92 (d, J=8.7 Hz, 2H), 6.78 (d, J=15.7 Hz, 1H), 4.63-4.56 (m, 1H), 4.19 (q, J=7.0 Hz, 2H), 4.11-4.00 (m, 5H), 3.42-3.30 (m, 4H), 2.82 (s, 3H), 2.05-1.94 (m, 4H), 1.41-1.35 (m, 3H), 1.24 (t, J=7.0 Hz, 3H).

### Example 9 Synthesis of (E)-8-(4-((1-(2,2-difluoroethyl)piperidin-4-yl)oxy)-3-fluorostyryl)-1,3-diethyl-7-methyl-1H-purine-2,6(3H,7H)-dione

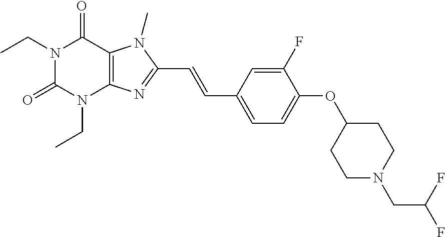 [SMILES: CCn1c(=O)c2c(nc(/C=C/c3ccc(OC4CCN(CC(F)F)CC4)c(F)c3)n2C)n(CC)c1=O]

### Step 1) Synthesis of tert-butyl 4-(2-fluoro-4-formylphenoxy)piperidine-1-carboxylate

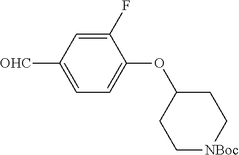 [SMILES: CC(C)(C)OC(=O)N1CCC(Oc2ccc(C=O)cc2F)CC1]

The title compound of this step was prepared by referring to the method described in step 2 of example 1, i.e., tert-butyl 4-(p-toluenesulfonyloxy) piperidine-1-carboxylate (5.0 g, 14.07 mmol) and 3-fluoro-4-hydroxybenzaldehyde (2.56 g, 18.3 mmol) were reacted in N,N-dimethylformamide (30 mL), and the crude product was purified by silica gel column chromatography (PE/EtOAc (v/v)=10/1) to give the title compound as a light yellow solid (4.53 g, 99%).

MS (ESI, pos. ion) m/z: 268.0 [M+H−56]⁺;

¹H NMR (400 MHz, CDCl₃) δ (ppm) 9.86 (d, J=1.9 Hz, 1H), 7.62 (d, J=8.9 Hz, 2H), 7.10 (t, J=7.9 Hz, 1H), 4.65 (tt, J=6.8, 3.3 Hz, 1H), 3.76-3.67 (m, 2H), 3.45-3.36 (m, 2H), 2.01-1.93 (m, 2H), 1.87-1.80 (m, 2H), 1.48 (s, 9H).

### Step 2) Synthesis of (E)-3-(4-((1-(tert-butoxycarbonyl)piperidin-4-yl)oxy)-3-fluorophenyl)acrylic acid

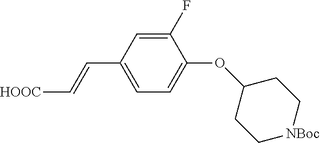 [SMILES: CC(C)(C)OC(=O)N1CCC(Oc2ccc(/C=C/C(=O)O)cc2F)CC1]

The title compound in this step was prepared by referring to the method described in step 3 of Example 1, i.e., 4-(2-fluoro-4-formylphenoxy)piperidine-1-carboxylate (4.5 g, 13.9 mmol), malonic acid (2.2 g, 21.2 mmol) were reacted in pyridine (10 mL), and the crude product was purified by silica gel column chromatography (PE/EtOAc (v/v)=3/1) to give the title compound as a white solid (4.0 g, 79%).

MS (ESI, pos. ion) m/z: 310.1 [M+H−56]⁺;

¹H NMR (400 MHz, CDCl₃) δ (ppm) 8.68 (s, 1H), 7.67 (d, J=15.9 Hz, 1H), 7.32 (dd, J=11.9, 1.7 Hz, 1H), 7.26 (d, J=8.5 Hz, 1H), 7.00 (t, J=8.4 Hz, 1H), 6.33 (d, J=15.9 Hz, 1H), 4.59-4.52 (m, 1H), 3.77-3.68 (m, 2H), 3.42-3.34 (m, 2H), 2.00-1.90 (m, 2H), 1.82 (dt, J=13.5, 7.2 Hz, 2H), 1.49 (s, 9H).

### Step 3) Synthesis of (E)-tert-butyl 4-(4-(3-((6-amino-1,3-diethyl-2,4-dioxo-1,2,3,4-tetrahydropyrimidine-5-yl)amino)-3-oxoprop-1-en-1-yl)-2-fluorophenoxy)piperidine-1-carboxylate

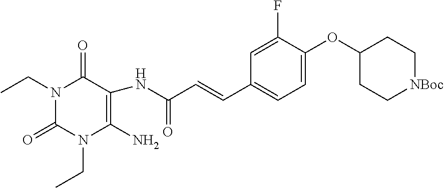 [SMILES: CCn1c(N)c(NC(=O)/C=C/c2ccc(OC3CCN(C(=O)OC(C)(C)C)CC3)c(F)c2)c(=O)n(CC)c1=O]

The title compound of this step was prepared by referring to the method described in step 4 of example 1, i.e., 5,6-diamino-1,3-diethylpyrimidine-2,4(1H,3H)-dione hydrochloride (3.3 g, 14 mmol), (E)-3-(4-((1-(tert-butoxycarbonyl)piperidin-4-yl)oxy)-3-fluorophenyl)acrylic acid (4 g, 10.9 mmol), HATU (5.3 g, 13.2 mmol) and N,N-diisopropyl ethylamine (7.2 mL, 41.1 mmol) were reacted in dichloromethane (40 mL) to prepare it. The crude product was purified by silica gel column chromatography (dichloromethane/methanol (v/v)=20/1) to give the title compound as a yellow solid (5.9 g, 99%).

MS (ESI, pos. ion) m/z: 546.2 [M+H]⁺;

¹H NMR (400 MHz, CDCl₃) δ (ppm) 7.73 (s, 1H), 7.50 (d, J=15.5 Hz, 1H), 7.22 (t, J=8.7 Hz, 2H), 6.97 (t, J=8.5 Hz, 1H), 6.59 (d, J=15.5 Hz, 1H), 5.72 (s, 2H), 4.56-4.49 (m, 1H), 3.77-3.68 (m, 4H), 3.40-3.32 (m, 2H), 3.23-3.15 (m, 2H), 1.93 (d, J=3.7 Hz, 2H), 1.84-1.75 (m, 2H), 1.48 (s, 9H), 1.32 (t, J=7.1 Hz, 3H), 1.19 (t, J=7.0 Hz, 3H).

### Step 4) Synthesis of (E)-tert-butyl 4-(4-(2-(1,3-diethyl-2,6-dioxo-2,3,6,7-tetrahydro-1H-purin-8-yl)vinyl)-2-fluorophenoxy)piperidine-1-carboxylate

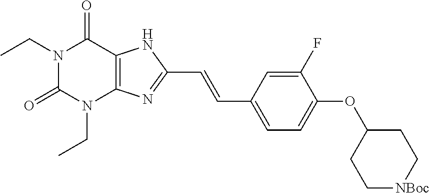 [SMILES: CCn1c(=O)c2[nH]c(/C=C/c3ccc(OC4CCN(C(=O)OC(C)(C)C)CC4)c(F)c3)nc2n(CC)c1=O]

The title compound of this step was prepared by referring to the method described in step 5 of example 1, i.e., (E)-tert-butyl 4-(4-(3-((6-amino-1,3-diethyl-2,4-dioxo-1,2,3,4-tetrahydropyrimidine-5-yl)amino)-3-oxoprop-1-en-1-yl)-2-fluorophenoxy)piperidine-1-carboxylate (5.9 g, 10.8 mmol) and sodium hydroxide (1.54 g, 38.6 mmol) were reacted in methanol (20 mL) and water (5 mL), and the crude product was purified by silica gel column chromatography (dichloromethane/methanol (v/v)=50/1) to give the title compound as a white solid (3.78 g, 65.1%).

MS (ESI, pos. ion) m/z: 528.2 [M+H]⁺;

¹H NMR (400 MHz, CDCl₃) δ (ppm) 12.83 (s, 1H), 7.69 (d, J=16.2 Hz, 1H), 7.35 (d, J=12.1 Hz, 1H), 7.25 (s, 1H), 7.05-6.97 (m, 2H), 4.59-4.52 (m, 1H), 4.31-4.21 (m, 4H), 3.79-3.71 (m, 2H), 3.43-3.35 (m, 2H), 1.99-1.92 (m, 2H), 1.87-1.78 (m, 2H), 1.50 (s, 9H), 1.43 (t, J=7.1 Hz, 3H), 1.37 (t, J=7.0 Hz, 3H).

### Step 5) Synthesis of (E)-tert-butyl 4-(4-(2-(1,3-diethyl-7-methyl-2,6-dioxo-2,3,6,7-tetrahydro-1H-purin-8-yl)vinyl)-2-fluorophenoxy)piperidine-1-carboxylate

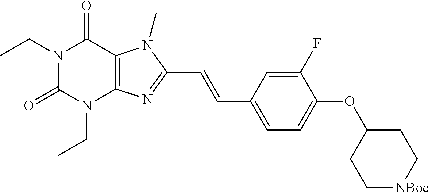 [SMILES: CCn1c(=O)c2c(nc(/C=C/c3ccc(OC4CCN(C(=O)OC(C)(C)C)CC4)c(F)c3)n2C)n(CC)c1=O]

The title compound of this step was prepared by referring to the method described in step 6 of example 1, i.e., (E)-tert-butyl 4-(4-(2-(1,3-diethyl-2,6-dioxo-2,3,6,7-tetrahydro-1H-purin-8-yl)vinyl)-2-fluorophenoxy)piperidine-1-carboxylate (3.7 g, 7.0 mmol), methyl iodide (0.9 mL, 14 mmol) and cesium carbonate (5.7 g, 18 mmol) were reacted in N,N-dimethylformamide (30 mL), and the crude product was purified by silica gel column chromatography (PE/EtOAc (v/v)=5/1) to give the title compound as a light yellow solid (2.2 g, 58%).

MS (ESI, pos. ion) m/z: 542.2 [M+H]⁺;

¹H NMR (400 MHz, CDCl₃) δ (ppm) 7.71 (d, J=15.7 Hz, 1H), 7.37 (dd, J=12.1, 1.7 Hz, 1H), 7.27 (s, 1H), 7.02 (t, J=8.4 Hz, 1H), 6.79 (d, J=15.7 Hz, 1H), 4.58-4.51 (m, 1H), 4.22 (q, J=7.0 Hz, 2H), 4.12 (q, J=7.0 Hz, 2H), 4.07 (s, 3H), 3.79-3.70 (m, 2H), 3.42-3.34 (m, 2H), 2.01-1.91 (m, 2H), 1.83 (dt, J=13.6, 7.3 Hz, 2H), 1.49 (s, 9H), 1.40 (t, J=7.1 Hz, 3H), 1.27 (d, J=7.0 Hz, 3H).

### Step 6 Synthesis of (E)-1,3-diethyl-8-(3-fluoro-4-(piperidin-4-yloxy)styryl)-7-methyl-1H-purine-2,6(3H,7H)-dione

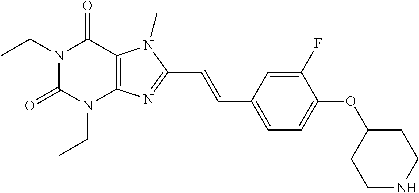 [SMILES: CCn1c(=O)c2c(nc(/C=C/c3ccc(OC4CCNCC4)c(F)c3)n2C)n(CC)c1=O]

The title compound of this step was prepared by referring to the method described in step 7 of example 1, i.e., (E)-tert-butyl 4-(4-(2-(1,3-diethyl-7-methyl-2,6-dioxo-2,3,6,7-tetrahydro-1H-purin-8-yl)vinyl)-2-fluorophenoxy)piperidine-1-carboxylate (2.2 g, 4.1 mmol) and methanesulfonic acid (0.8 mL, 12 mmol) were reacted in dichloromethane (15 mL) to give the title compound as a light yellow solid (1.7 g, 95%).

MS (ESI, pos. ion) m/z: 442.2 [M+H]⁺;

¹H NMR (400 MHz, CDCl₃) δ (ppm) 7.75 (d, J=15.7 Hz, 1H), 7.53 (d, J=8.7 Hz, 2H), 6.95 (d, J=8.7 Hz, 2H), 6.78 (d, J=15.7 Hz, 1H), 4.50-4.43 (m, 1H), 4.23 (q, J=7.1 Hz, 2H), 4.10 (q, J=7.0 Hz, 2H), 4.06 (s, 3H), 3.22-3.15 (m, 2H), 2.80 (ddd, J=12.3, 9.1, 3.0 Hz, 2H), 2.07 (d, J=6.5 Hz, 2H), 1.78-1.68 (m, 2H), 1.40 (t, J=7.1 Hz, 3H), 1.28 (t, J=7.0 Hz, 3H).

### Step 7 Synthesis of (E)-8-(4-((1-(2,2-difluoroethyl)piperidin-4-yl)oxy)-3-fluorostyryl)-1,3-diethyl-7-methyl-1H-purine-2,6(3H,7H)-dione

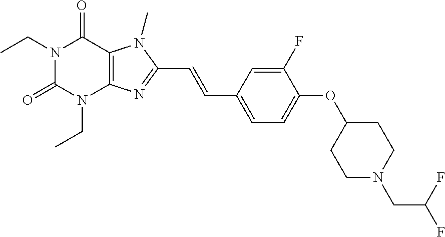 [SMILES: CCn1c(=O)c2c(nc(/C=C/c3ccc(OC4CCN(CC(F)F)CC4)c(F)c3)n2C)n(CC)c1=O]

The title compound of this step was prepared by referring to the method described in example 4, i.e., (E)-1,3-diethyl-8-(3-fluoro-4-(piperidin-4-yloxy)styryl)-7-methyl-1H-purine-2,6(3H,7H)-dione (0.5 g, 1.13 mmol), potassium carbonate (474 mg, 3.4 mmol), sodium iodide (170 mg, 1.13 mmol) and 2,2-difluoroethyl p-toluenesulfonate (803 mg, 3.4 mmol) were reacted in acetonitrile (20 mL), and the crude product was purified by silica gel column chromatography (PE/EtOAc (v/v)=1/1) to give the title compound as an off-white solid (0.46 g, 80.3%).

MS (ESI, pos. ion) m/z: 506.1 [M+H]⁺;

¹H NMR (400 MHz, CDCl₃) δ (ppm) 7.68 (d, J=15.7 Hz, 1H), 7.34 (dd, J=12.1, 1.5 Hz, 1H), 7.24 (s, 1H), 6.98 (t, J=8.4 Hz, 1H), 6.76 (d, J=15.7 Hz, 1H), 5.89 (tt, J=56.0, 4.2 Hz, 1H), 4.44-4.37 (m, 1H), 4.20 (q, J=7.1 Hz, 2H), 4.08 (q, J=7.1 Hz, 2H), 4.05 (s, 3H), 2.90-2.85 (m, 2H), 2.77 (td, J=15.0, 4.3 Hz, 2H), 2.57-2.52 (m, 2H), 2.05-2.01 (m, 2H), 1.96-1.90 (m, 2H), 1.37 (t, J=7.1 Hz, 3H), 1.26 (t, J=7.0 Hz, 3H).

### Example 10 Synthesis of (E)-8-(4-((1-acetylpiperidin-4-yl)oxy)-3-fluorostyryl)-1,3-diethyl-7-methyl-1H-purine-2,6(3H,7H)-dione

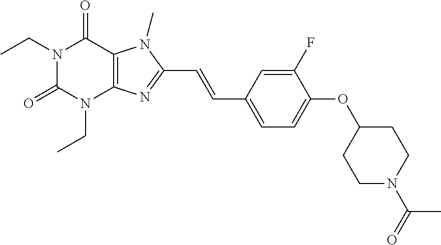 [SMILES: CCn1c(=O)c2c(nc(/C=C/c3ccc(OC4CCN(C(C)=O)CC4)c(F)c3)n2C)n(CC)c1=O]

The title compound of this step was prepared by referring to the method described in example 6, i.e., (E)-1,3-diethyl-8-(3-fluoro-4-(piperidin-4-yloxy)styryl)-7-methyl-1H-purine-2,6(3H,7H)-dione (0.5 g, 1.13 mmol), triethylamine (0.48 mL, 3.4 mmol) and acetic anhydride (0.21 mL, 2.2 mmol) were reacted in dichloromethane (8 mL), and the crude product was purified by silica gel column chromatography (PE/EtOAc (v/v)=1/1) to give the title compound as an off-white solid (0.52 g, 94.9%).

MS (ESI, pos. ion) m/z: 484.4 [M+H]⁺;

¹H NMR (400 MHz, CDCl₃) δ (ppm) 7.70 (d, J=15.7 Hz, 1H), 7.37 (dd, J=12.1, 1.7 Hz, 1H), 7.29 (d, J=5.9 Hz, 1H), 7.02 (t, J=8.4 Hz, 1H), 6.79 (d, J=15.7 Hz, 1H), 4.65-4.58 (m, 1H), 4.22 (q, J=7.0 Hz, 2H), 4.12 (q, J=7.0 Hz, 2H), 4.07 (s, 3H), 3.83-3.66 (m, 3H), 3.49-3.41 (m, 1H), 2.14 (s, 3H), 1.98-1.84 (m, 4H), 1.39 (t, J=7.1 Hz, 3H), 1.27 (t, J=7.0 Hz, 3H).

### Example 11 Synthesis of (E)-8-(4-((1-(cyclopropenyl)piperidin-4-yl)oxy)-3-fluorostyryl)-1,3-diethyl-7-methyl-1H-purine-2,6(3H,7H)-dione

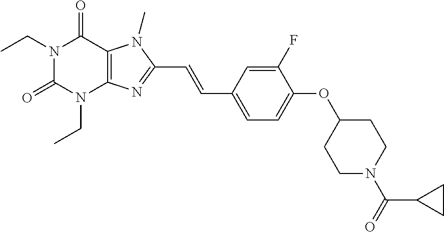 [SMILES: CCn1c(=O)c2c(nc(/C=C/c3ccc(OC4CCN(C(=O)C5CC5)CC4)c(F)c3)n2C)n(CC)c1=O]

The title compound of this step was prepared by referring to the method described in example 7, i.e., (E)-1,3-diethyl-8-(3-fluoro-4-(piperidin-4-yloxy)styryl)-7-methyl-1H-purine-2,6(3H,7H)-dione (0.5 g, 1.13 mmol), triethylamine (0.64 mL, 4.53 mmol) and cyclopropylformyl chloride (0.21 mL, 2.3 mmol) were reacted in dichloromethane (8 mL), and the crude product was purified by silica gel column chromatography (PE/EtOAc (v/v)=1/1) to give the title compound as an off-white solid (0.51 g, 88.4%).

MS (ESI, pos. ion) m/z: 510.4 [M+H]⁺;

¹H NMR (400 MHz, CDCl₃) δ (ppm) 7.71 (d, J=15.7 Hz, 1H), 7.38 (dd, J=12.1, 1.7 Hz, 1H), 7.29 (d, J=6.0 Hz, 1H), 7.04 (t, J=8.4 Hz, 1H), 6.80 (d, J=15.7 Hz, 1H), 4.67-4.61 (m, 1H), 4.23 (q, J=7.0 Hz, 2H), 4.12 (dd, J=13.7, 7.0 Hz, 2H), 4.07 (s, 3H), 3.91-3.88 (m, 2H), 3.73-3.65 (m, 2H), 2.03-1.87 (m, 4H), 1.80 (ddd, J=12.6, 8.1, 4.7 Hz, 1H), 1.40 (t, J=7.1 Hz, 3H), 1.28 (t, J=7.0 Hz, 3H), 1.02 (brs, 2H), 0.79 (dd, J=7.9, 2.9 Hz, 2H).

Biological Assay

### Example A: Evaluation of the Antagonizing Effect of the Compound of the Invention on Human Adenosine A_{2A} Receptor

Test Method:

The experimental system uses human recombinant adenosine A_{2A} receptor, which is stably expressed in HEK-293 cell line. HEK-293 cells were inoculated into culture plate at a density of 1.25×10⁵ cells/mL. The medium was changed by modified HBSS solution with pH 7.4, different concentrations of test compounds (10 M, 1 μM, 0.1 M, 10 nM, 1 nM, 0.1 nM) and solvents (PBS with 0.1% DMSO) were added, and the plate was incubated at 37° C. for 10 min. cAMP (cyclic adenosine monophosphate) concentration in culture medium was detected by TR-FRET method. NECA was used as a positive control in this experiment, (1) test compounds alone were used to stimulate cells to produce a certain amount of cAMP, and then NECA (0.1 μM) alone was used to stimulate cells to produce a certain amount of cAMP. A test compound is considered to have agonistic activity on adenosine A_{2A} receptor if the test compound alone stimulates cells to produce cAMP more than 50% of the amount of cAMP produced by NECA (0.1 μM) alone. (2) All cells were first incubated with test compounds and then stimulated with NECA (3 nM). In addition, NECA (3 nM) alone was used to stimulate cells to produce a certain amount of cAMP. If the amount of cAMP produced by cells incubated with the test compound and then stimulated with NECA (3 nM) was less than 50% of the amount of cAMP produced by NECA (3 nM) alone, i.e., the inhibition rate of the test compound on the amount of cAMP produced by NECA (3 nM) is more than 50%, it is indicated that the test compound has antagonistic activity on adenosine A_{2A} receptor. IC₅₀ was calculated using MathIQ™ (ID Business Solutions Ltd., UK) through nonlinear, least squares regression equation analysis. The experimental results were shown in Table A.

TABLE A
Experimental results of antagonizing effect of the compound of the
invention on human adenosine A_{2A} receptor.

| Example No. | IC₅₀ (μM) |
|---|---|
| Example 1 | 0.55 |
| Example 3 | 1.48 |
| Example 6 | 0.35 |
| Example 8 | 0.75 |

The experimental results show that the compound of the invention has a strong antagonistic effect on adenosine A_{2A} receptor.

### Example B: Pharmacokinetic Evaluation after Administering a Certain Amount of the Compound of the Invention by Intravenous or Gavage to Rats

### (1) Test Animal

The test animals are rats, as shown in Table 1:

TABLE 1

| Germline | Grade | Sex | Weight | Week age | Source |
|---|---|---|---|---|---|
| SD rats | Cleaning | male | 180~220 g | 8 weeks | Cavins, Changzhou |
| | class | | | | |

### (2) Analytical Method

The LC-MS/MS system for analysis includes the Agilent 1200 series vacuum degasser, a quaternary pump, an orifice plate sampler, a thermostatted column oven, and an API4000Qtrap triple quadrupole mass spectrometer with an electrospray ionization source (ESI). Quantitative analysis is performed in MRM mode, where the source parameters of MRM conversion are shown in Table 2:

TABLE 2

| | | |
|---|---|---|
| Curtain gas/CUR: | 30 | psi |
| Atomizing gas/GS1: | 55 | psi |
| Auxiliary heating gas/GS2: | 60 | psi |
| Ion transmission voltage/IS: | 5000 | V |
| Atomization temperature/TEM: | 500° | C. |

Waters ACQUITY UPLC CSH C18, 2.1×50 mm, 1.7 μm column was used for analysis, and 0.8 μL of sample was injected. Analytical conditions: mobile phase was H₂O+2 mM HCOONH₄ (ammonium formate)+0.1% FA (formic acid) (mobile phase A) and MeOH (methanol)+2 mM HCOONH₄ (ammonium formate)+0.1% FA (formic acid) (mobile phase B). Flow rate was 0.7 mL/min. The column temperature was 40° C., and the mobile phase gradient was shown in Table 3:

TABLE 3

| Time | Gradient of mobile phase B |
|---|---|
| 0.4 min | 10% |
| 0.6 min | 95% |
| 1.6 min | 95% |
| 1.61 min | 10% |
| 2.50 min | Stop |

(3) Test Method:

The pharmacokinetic evaluation of the compound of the present invention on rats in vivo was performed as follows:

The experiment was divided into two groups: one was administered by intravenous injection, and the other was administered by intragastric administration. The compound of the present invention was administered to a test animal (overnight fast for 12 hours) in the form of 10% DMA (heating)+60% PEG400+30% saline solution. For the group of intravenous administration, the administration dose was 1 mg/kg, and vein blood samples (0.3 mL) were collected at the time points of 0.083, 0.25, 0.5, 1.0, 2.0, 5.0, 7.0 and 24 h after administration. EDTA-K₂ as anticoagulant was added into the blood vessel. The plasma solutions were collected by centrifuging each blood sample at 3,000 rpm or 4,000 rpm for 10 minutes and kept at −20° C. or −70° C. For the group of intragastric administration, the administration dose was 5 mg/kg, and vein blood samples (0.3 mL) were collected at the time points of 0.25, 0.5, 1.0, 2.0, 5.0, 7.0 and 24 h after administration. EDTA-K₂ as anticoagulant was added into the blood vessel. The plasma solutions were collected by centrifuging each blood sample at 3,000 rpm or 4,000 rpm for 10 minutes and kept at −20° C. or −70° C.

20 μL of plasma was taken, 120 μL of IS working solution was added to the plasma, and the plasma was vortexed for 2 min. The mixed solution was then centrifuged at 12,000 rpm for 2 min. 100 μL of the supernatant was taken, 110 μL of MeOH/H₂O (v/v=1/1) was added, and the solution was vortexed for 2 minutes, then 5 μL was taken for injection into the LC-MS/MS system. LC-MS/MS method was used to detect the concentration of target compounds, and non-compartment model was used to calculate pharmacokinetic parameters. The experimental results were shown in Table B. The analysis results show that the compound of the invention has good pharmacokinetic properties in rats.

TABLE B
Pharmacokinetic parameters of the compound of the present
invention in rats

| | Example 6 | | Example 8 | |
|---|---|---|---|---|
| PK | Intravenous | | Intravenous | |
| parameters | injection | Gavage | injection | Gavage |
| Dosage | 1 | 5 | 1 | 5 |
| (mg/kg) | | | | |
| Tₘₐₓ (h) | 0.083 | 1.5 | 0.083 | 1.67 |
| Cₘₐₓ (ng/ml) | 1610 | 2930 | 817 | 2030 |
| AUCₗₐₛₜ | 6230 | 36900 | 1810 | 11900 |
| (h*ng/ml) | | | | |
| AUC_{INF} | 6290 | 40400 | 1830 | 11900 |
| (h*ng/ml) | | | | |
| MRT_{INF} (h) | 4.37 | 9.22 | 3.1 | 4.07 |
| T_{1/2} (h) | 3.82 | 6.96 | 4.64 | 2.8 |
| Cl | 2.65 | / | 9.1 | / |
| (ml/min/kg) | | | | |
| Vₛₛ (l/kg) | 0.696 | / | 1.69 | / |
| F (%) | / | 118.4 | / | 131.5 |

### Example C: Pharmacokinetic Evaluation after Administering a Certain Amount of the Compound of the Invention by Intravenous or Gavage to Mice/Dogs

(1) Test Animals:

The test animals are mice and/or dogs. The specific conditions are shown in Table 4:

TABLE 4

| Germline | Grade | Sex | Quantity | Weight | | Week age | Source |
|---|---|---|---|---|---|---|---|
| ICR | Cleaning | male | 6 | 18~22 | g | 8 weeks | Cavins, Changzhou |
| mice | class | | | | | | |
| Beagles | Ordinary | male | 4 | 6~8 | kg | June to | Beijing Marshall |
| | | | | | | August | Biotechnology Co., Ltd. |

(2) Analytical Method:

The LC-MS/MS system for analysis includes the Agilent 1200 series vacuum degasser, a quaternary pump, an orifice plate sampler, a thermostatted column oven, and an API4000Qtrap triple quadrupole mass spectrometer with an electrospray ionization source (ESI). Quantitative analysis is performed in MRM mode, where the source parameters of MRM conversion are shown in Table 5:

TABLE 5

| | | |
|---|---|---|
| Curtain gas/CUR: | 20 | psi |
| Atomizing gas/GS1: | 60 | psi |
| Auxiliary heating gas/GS2: | 70 | psi |
| Ion transmission voltage/IS: | 4500 | V |
| Atomization temperature/TEM: | 550° | C. |

A waters xbridge C18 UPLC, 2.1×50 mm, 3.5 μM column was used for analysis and 0.5 μL of sample was injected. Analytical conditions: mobile phase was H₂O+2 mM HCOONH₄ (ammonium formate)+0.1% FA (formic acid) (mobile phase A) and MeOH (methanol)+2 mM HCOONH₄ (ammonium formate)+0.1% FA (formic acid) (mobile phase B). The flow rate was 0.7 mL/min. The mobile phase gradient was shown in Table 6:

TABLE 6

| Time | Gradient of mobile phase B |
|---|---|
| 0.3 min | 20% |
| 0.7 min | 95% |
| 1.8 min | 95% |
| 1.81 min | 20% |
| 2.8 min | Stop |

(3) Test Method

### 1) The Pharmacokinetic Evaluation of the Compound of the Present Invention on Mice In Vivo was Performed as Follows:

The experiment was divided into two groups: one was administered by intravenous injection, and the other was administered by intragastric administration. The compound of the present invention was administered to a test animal in the form of 10% DMA+60% PEG400+30% saline solution. For the group of intravenous administration, the administration dose was 2 mg/kg, and the vein blood samples (0.3 mL) were taken at the time points of 0.083, 0.25, 0.5, 1.0, 2.0, 5.0, 7.0 and 24 h after administration. Each blood sample was centrifuged at 3,000 rpm or 4,000 rpm for 10 minutes. The plasma solutions were collected and anticoagulant was EDTA-K₂. The plasma solutions were then kept at −20° C. or −70° C. For the group of intragastric administration, the administration dose was 5 mg/kg, and vein blood samples (0.3 mL) were collected at the time points of 0.083, 0.25, 0.5, 1.0, 2.0, 5.0, 7.0 and 24 h after administration. EDTA-K₂ as anticoagulant was added into the blood vessel. The plasma solutions were collected by centrifuging each blood sample at 3,000 rpm or 4,000 rpm for 10 minutes and kept at −20° C. or −70° C.

10 μL of plasma was taken, 130 μL of IS working solution was added to the plasma, and the plasma was vortexed for 5 min. The mixed solution was then centrifuged at 4,000 rpm for 5 min. 100 μL of the supernatant was taken, 150 μL of H₂O was added, and the solution was vortexed for 2 minutes, then 2.5 μL was taken for injection into the LC-MS/MS system. LC-MS/MS method was used to detect the concentration of target compounds, and non-compartment model was used to calculate pharmacokinetic parameters. The analysis results show that the compound of the invention has good pharmacokinetic properties in mice.

### 2) The Pharmacokinetic Evaluation of the Compound of the Present Invention on Beagles In Vivo was Performed as Follows:

The experiment was divided into two groups: one was administered by intravenous injection, and the other was administered by intragastric administration. The compound of the present invention was administered to test animals in the form of 10% DMA+60% PEG400+30% saline solution. For the group of intravenous administration, the administration dose was 1 mg/kg, and the vein blood samples (0.3 mL) were taken at the time points of 0.083, 0.25, 0.5, 1.0, 2.0, 4.0, 8.0 and 24 h after administration. Each blood sample was centrifuged at 3,000 rpm or 4,000 rpm for 10 minutes. The plasma solutions were collected and anticoagulant was EDTA-K₂. The plasma solutions were then kept at −20° C. or −70° C. For the group of intragastric administration, the administration dose was 5 mg/kg, and vein blood samples (0.3 mL) were taken at the time points of 0.25, 0.5, 1.0, 2.0, 5.0, 7.0 and 24 h after administration. Each blood sample was centrifuged at 3,000 rpm or 4,000 rpm for 10 minutes. The plasma solutions were collected and anticoagulant was EDTA-K₂. The plasma solutions were then kept at −20° C. or −70° C.

10 μL of plasma was taken, 120 μL of IS working solution was added to the plasma, and the plasma was vortexed for 2 min. The mixed solution was then centrifuged at 12,000 rpm for 2 min. 80 μL of the supernatant was taken, 140 μL of MeOH/H₂O (v/v=1/1) was added, and the solution was vortexed for 2 minutes, then 1.0 μL was taken for injection into the LC-MS/MS system. LC-MS/MS method was used to detect the concentration of target compounds, and non-compartment model was used to calculate pharmacokinetic parameters. The experimental results were shown in Table C. The analysis results show that the compound of the invention has good pharmacokinetic properties in beagles.

TABLE C
Pharmacokinetic parameters of the compound of the present
invention in beagles

| | Example 6 | | Example 8 | |
|---|---|---|---|---|
| PK | Intravenous | | Intravenous | |
| parameters | injection | Gavage | injection | Gavage |
| Dosage | 0.5 | 2.5 | 0.5 | 2.5 |
| (mg/kg) | | | | |
| Tₘₐₓ (h) | 0.25 | 2.67 | 0.083 | 0.667 |
| Cₘₐₓ (ng/ml) | 634 | 3320 | 775 | 1720 |
| AUCₗₐₛₜ | 5830 | 47200 | 4990 | 22700 |
| (h*ng/ml) | | | | |
| AUC_{INF} | 6110 | 56500 | 6310 | 31800 |
| (h*ng/ml) | | | | |
| MRT_{INF} (h) | 7.37 | 12.5 | 14.4 | 18.1 |
| T_{1/2} (h) | 5.81 | 9.11 | 9.43 | 11.5 |
| Cl | 1.36 | / | 1.32 | / |
| (ml/min/kg) | | | | |
| Vₛₛ (l/kg) | 0.604 | / | 1.14 | / |
| F (%) | / | 170.5 | / | 91.1 |

Reference throughout this specification to “an embodiment”, “some embodiments”, “one embodiment”, “another example”, “an example”, “a specific example” or “some examples” means that a particular feature, structure, material, or characteristic described in connection with the embodiment or example is included in at least one embodiment or example of the present disclosure. Thus, the appearances of the phrases such as “in some embodiments”, “in one embodiment”, “in an embodiment”, “in another example”, “in an example”, “in a specific example” or “in some examples” in various places throughout this specification are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1.-20. (canceled)

21. A compound having Formula (I) or a stereoisomer, a tautomer, an N-oxide, a hydrate, a solvate, a metabolite, a pharmaceutically acceptable salt or a prodrug thereof, 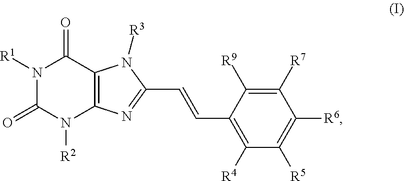
wherein
each of R¹, R² and R³ is independently H, D, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, hydroxyl substituted C₁-C₆ alkyl, C₃-C₈ cycloalkyl, 3-8 membered heterocyclyl, C₆-C₁₀ aryl or 5-10 membered heteroaryl;
each of R⁴, R⁵ and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxyl substituted C₁-C₆ alkyl, C₃-C₈ cycloalkyl, 3-8 membered heterocyclyl, C₆-C₁₀ aryl or 5-10 membered heteroaryl;
R⁶ is —O—R⁰, R⁷ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxyl substituted C₁-C₆ alkyl, C₃-C₈ cycloalkyl, 3-8 membered heterocyclyl, C₆-C₁₀ aryl, 5-10 membered heteroaryl or —O—R⁰; or
R⁶ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₆ alkyl), —C(═O)—(C₁-C₆ alkoxy), C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ alkylthio, C₁-C₆ alkylamino, hydroxy substituted C₁-C₆ alkyl, C₃-C₈ cycloalkyl, 3-8 membered heterocyclyl, C₆-C₁₀ aryl or 5-10 membered heteroaryl, R⁷ is —O—R⁰;
R⁰ is 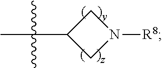
y is 1, 2, or 3;
z is 1, 2, or 3; and
R⁸ is H, D, —COOH, —C(═O)NH₂, —C(═O)NHCH₃, —C(═O)N(CH₃)₂, —C(═O)—(C₁-C₆ alkyl), —C(═O)—(C₃-C₈ cycloalkyl), —C(═O)-(3-8 membered heterocyclyl), —C(═O)—(C₆-C₁₀ aryl), —C(═O)-(5-10 membered heteroaryl), —C(═O)—(C₁-C₆ alkoxy), —S(═O)₂—(C₁-C₆ alkyl), —S(═O)₂—(C₃-C₈ cycloalkyl), —S(═O)₂-(3-8-membered heterocyclyl), —S(═O)₂—(C₆-C₁₀ aryl), —S(═O)₂-(5-10 membered heteroaryl), C₁-C₆ alkyl, C₂-C₆ alkenyl, C₂-C₆ alkynyl, C₁-C₆ haloalkyl, hydroxy substituted C₁-C₆ alkyl, C₃-C₈ cycloalkyl, 3-8 membered heterocyclyl, C₆-C₁₀ aryl or 5-10 membered heteroaryl.

22. The compound of claim 21, wherein R⁰ is

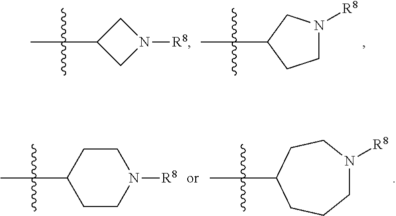

23. The compound of claim 21, wherein R⁸ is H, D, —COOH, —C(═O)NH₂, —C(═O)NHCH₃, —C(═O)N(CH₃)₂, —C(═O)—(C₁-C₄ alkyl), —C(═O)—(C₃-C₆ cycloalkyl), —C(═O)-(3-6 membered heterocyclyl), —C(═O)—(C₆-C₁₀ aryl), —C(═O)-(5-10 membered heteroaryl), —C(═O)—(C₁-C₄ alkoxy), —S(═O)₂—(C₁-C₄ alkyl), —S(═O)₂—(C₃-C₆ cycloalkyl), —S(═O)₂-(3-6 membered heterocyclyl), —S(═O)₂—(C₆-C₁₀ aryl), —S(═O)₂-(5-10 membered heteroaryl), C₁-C₄ alkyl, C₂-C₄ alkenyl, C₂-C₄ alkynyl, C₁-C₄ haloalkyl, hydroxy substituted C₁-C₄ alkyl, C₃-C₆ cycloalkyl, 3-6 membered heterocyclyl, C₆-C₁₀ aryl or 5-10 membered heteroaryl.

24. The compound of claim 21, wherein R⁸ is H, D, —COOH, —C(═O)NH₂, —C(═O)NHCH₃, —C(═O)N(CH₃)₂, —C(═O)—CH₃, —C(═O)—CH₂CH₃, 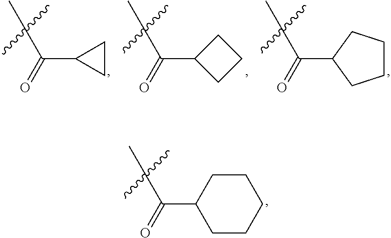
—C(═O)-(3-6 membered heterocyclyl), —C(═O)-Ph, —C(═O)-(5-6 membered heteroaryl), —C(═O)—OCH₃, —S(═O)₂—CH₃, —S(═O)₂—CH₂CH₃, —S(═O)₂—(C₃-C₆ cycloalkyl), —S(═O)₂-(3-6 membered heterocyclyl), —S(═O)₂-Ph, —S(═O)₂-(5-6 membered heteroaryl), methyl, ethyl, n-propyl, isopropyl, allyl, propenyl, propargyl, propynyl, —CHF₂, —CF₃, —CHFCH₂F, —CF₂CHF₂, —CH₂CHF₂, —CH₂CF₃, —CH₂CF₂CHF₂, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazoly, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl.

25. The compound of claim 21, wherein each of R¹, R² and R³ is independently H, D, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, hydroxyl substituted C₁-C₄ alkyl, C₃-C₆ cycloalkyl, 3-6 membered heterocyclyl, C₆-C₁₀ aryl or 5-10 membered heteroaryl;
each of R⁴, R⁵ and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxyl substituted C₁-C₄ alkyl, C₃-C₆ cycloalkyl, 3-6 membered heterocyclyl, C₆-C₁₀ aryl or 5-10 membered heteroaryl.

26. The compound of claim 21, wherein each of R¹, R² and R³ is independently H, D, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl;
each of R⁴, R⁵ and R⁹ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl.

27. The compound of claim 21, wherein R⁶ is —O—R⁰, R⁷ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxyl substituted C₁-C₄ alkyl, C₃-C₆ cycloalkyl, 3-6 membered heterocyclyl, C₆-C₁₀ aryl or 5-10 membered heteroaryl or —O—R⁰; or
R⁶ is H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—(C₁-C₄ alkyl), —C(═O)—(C₁-C₄ alkoxy), C₁-C₄ alkyl, C₂-C₄ alkenyl, C₂-C₄ alkynyl, C₁-C₄ haloalkyl, C₁-C₄ alkoxy, C₁-C₄ haloalkoxy, C₁-C₄ alkylthio, C₁-C₄ alkylamino, hydroxy substituted C₁-C₄ alkyl, C₃-C₆ cycloalkyl, 3-6 membered heterocyclyl, C₆-C₁₀ aryl or 5-10 membered heteroaryl, R⁷ is —O—R⁰.

28. The compound of claim 21, wherein R⁶ is —O—R⁰, R⁷ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl or —O—R⁰; or
R⁶ is independently H, D, F, Cl, Br, I, —CN, —NO₂, —NH₂, —OH, —SH, —COOH, —C(═O)NH₂, —C(═O)NHCH₃, —C(═O)N(CH₃)₂, —C(═O)—CH₃, —C(═O)—OCH₃, methyl, ethyl, n-propyl, isopropyl, allyl, propenyl, propargyl, propynyl, —CHF₂, —CF₃, —CHFCH₂F, —CF₂CHF₂, —CH₂CF₃, —CH₂CF₂CHF₂, methoxy, ethoxy, n-propyloxy, isopropyloxy, —OCHF₂, —OCF₃, —OCHFCH₂F, —OCF₂CHF₂, —OCH₂CF₃, —OCH₂CF₂CHF₂, methylthio, ethylthio, methylamino, dimethylamino, ethylamino, hydroxymethyl, 2-hydroxyethyl, cyclopropyl, cyclobutyl, cyclopentyl, cyclohexyl, azetidinyl, pyrrolidinyl, tetrahydrofuranyl, piperidinyl, piperazinyl, morpholinyl, phenyl, indenyl, naphthyl, pyrrolyl, pyrazolyl, imidazolyl, triazolyl, tetrazolyl, furyl, thienyl, thiazolyl, oxazolyl, pyridyl, pyrimidinyl, pyrazinyl, pyridazinyl, benzimidazolyl, indolyl or quinolinyl, R⁷ is —O—R⁰.

29. The compound of claim 21 having one of the following structures or a stereoisomer, a tautomer, an N-oxide, a hydrate, a solvate, a metabolite, a pharmaceutically acceptable salt or a prodrug thereof:

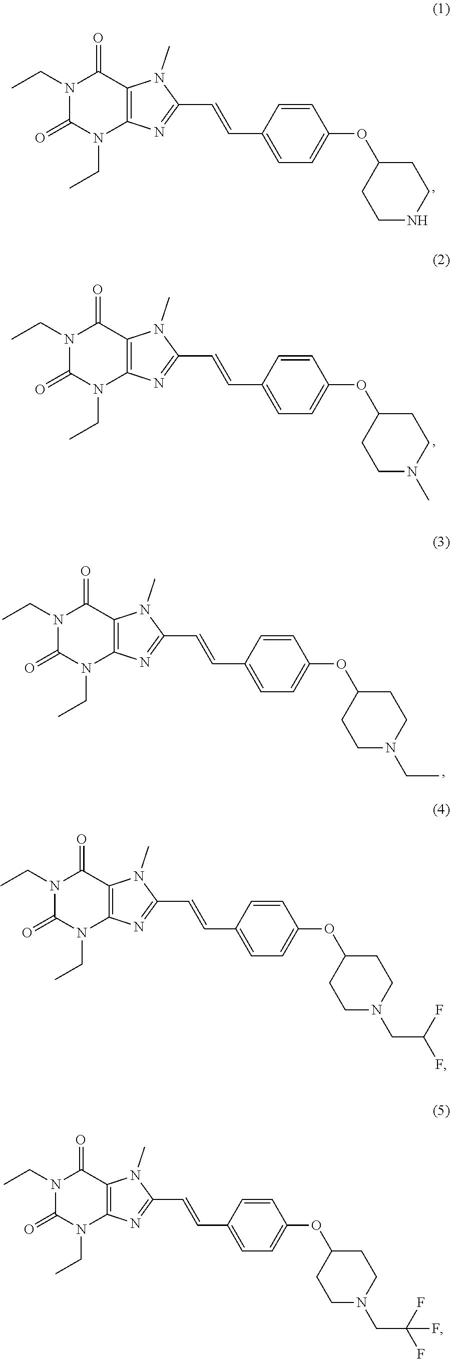

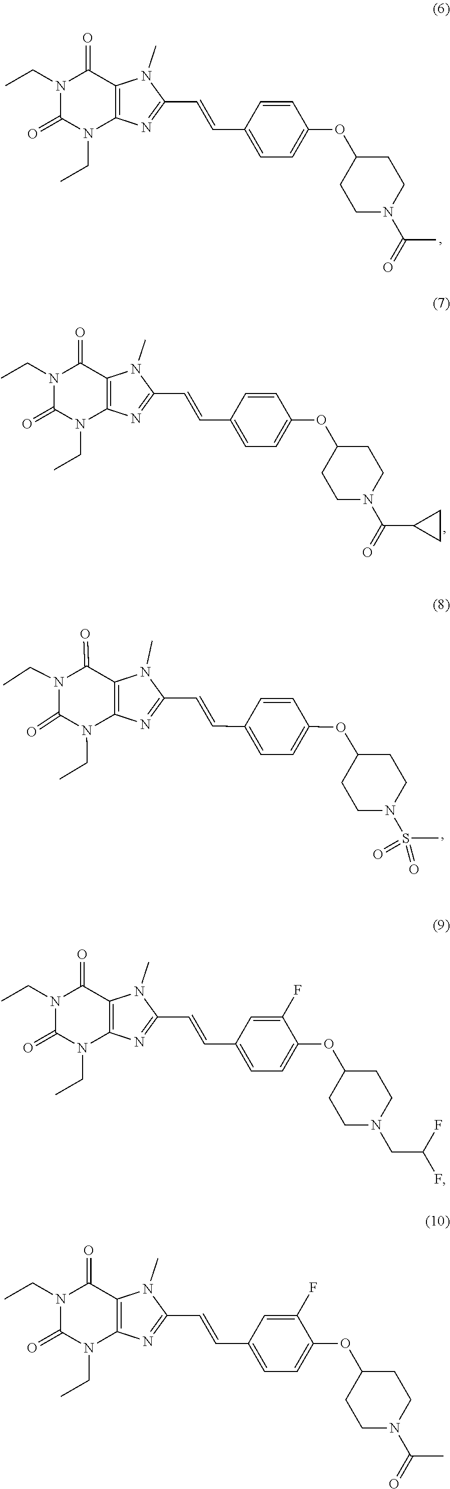

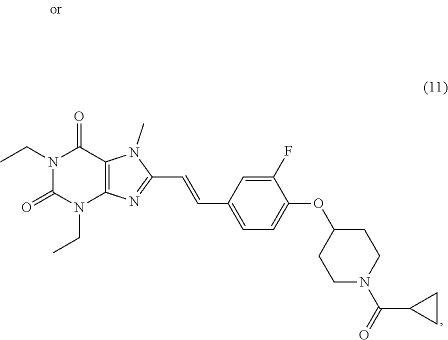 [SMILES: CCn1c(=O)c2c(nc(/C=C/c3ccc(OC4CCN(C(=O)C5CC5)CC4)c(F)c3)n2C)n(CC)c1=O]

30. A pharmaceutical composition comprising the compound of claim 21; and wherein the pharmaceutical composition optionally further comprising a pharmaceutically acceptable excipient, carrier, or adjuvant or any combination thereof.

31. A pharmaceutical composition comprising the compound of claim 29; and wherein the pharmaceutical composition optionally further comprising a pharmaceutically acceptable excipient, carrier, or adjuvant or any combination thereof.

32. The pharmaceutical composition according to claim 30 further comprising an additional therapeutic agent, wherein the additional therapeutic agent is monoamine oxidase type B inhibitor, dopamine agonist, anticholinergic drug, glutamate antagonist, levodopa or any combination thereof.

33. The pharmaceutical composition according to claim 31 further comprising an additional therapeutic agent, wherein the additional therapeutic agent is monoamine oxidase type B inhibitor, dopamine agonist, anticholinergic drug, glutamate antagonist, levodopa or any combination thereof.

34. A method of preventing, treating or lessening an adenosine A_{2A} receptor-related disease comprising administering a therapeutically effective amount of the compound of claim 21 to a subject.

35. The method of claim 34, wherein the adenosine A_{2A} receptor-related disease is Parkinson's disease, tumour, pain, depression, dementia, stroke, myocardial ischemia, asthma, alcohol withdrawal, dyskinesia syndrome, restless leg syndrome, dystonia, systemic stiffness, neurodegenerative disorders or osteoporosis.

36. A method of antagonizing adenosine A_{2A} receptor comprising administering a therapeutically effective amount of the compound of claim 21 to a subject.

37. A method of preventing, treating or lessening an adenosine A_{2A} receptor-related disease comprising administering a therapeutically effective amount of the pharmaceutical composition of claim 30 to a subject.

38. The method of claim 37, wherein the adenosine A_{2A} receptor-related disease is Parkinson's disease, tumour, pain, depression, dementia, stroke, myocardial ischemia, asthma, alcohol withdrawal, dyskinesia syndrome, restless leg syndrome, dystonia, systemic stiffness, neurodegenerative disorders or osteoporosis.

39. A method of antagonizing adenosine A_{2A} receptor comprising administering a therapeutically effective amount of the pharmaceutical composition of claim 30 to a subject.
